(19) United States
(12) Patent Application Publication
(10) Pub. No.: US 2010/0144738 A1
(43) Pub. Date: Jun. 10, 2010

(54) INHIBITORS OF C-MET AND USES THEREOF

(76) Inventors: William Bornmann, Missouri City, TX (US); David Maxwell, Pearland, TX (US); Yunming Ying, Austin, TX (US); Dongmei Han, San Mateo, CA (US); Zhenghong Peng, Missouri City, TX (US); Varsha Gandhi, Houston, TX (US); Christine Stellrecht, Friendswood, TX (US)

Correspondence Address: Nielsen IP Law LLC, 1177 West Loop South, Suite 1600, Houston, TX, 77027, US

(21) Appl. No.: 12/439,491

(22) PCT Filed: Aug. 29, 2007
(86) PCT No.: PCT/US2007/077117

§ 371 Date: Feb. 5, 2010

Related U.S. Application Data
(60) Provisional application No. 60/824,556, filed on Sep. 5, 2006.

(51) Int. Cl. A61K 31/522; C07D 473/00; C07D 487/04; A61K 31/5025; A61P 25/00

(52) U.S. Cl. 514/248; 544/276; 544/236; 514/263.23

(57) ABSTRACT

The present invention relates to compounds and methods use as inhibitors of c-Met. Certain compounds of the subject invention have the following structural formula (I). Other compounds of the subject invention have structural formulas as defined herein. Also disclosed herein are pharmaceutical compositions comprising the compounds of the subject invention.

### CROSS-REFERENCE TO RELATED APPLICATIONS

This application claims priority to U.S. Pat. App. Ser. No. 60/824,556 filed Sep. 5, 2006. The application is incorporated by reference herein in its entirety.

### STATEMENT REGARDING FEDERALLY SPONSORED RESEARCH OR DEVELOPMENT

The present invention has been funded in part by the U.S. Governmental Grant No. Leukemia Spore, M D Anderson, P50 CA100632.

### FIELD OF THE INVENTION

The present invention is directed to compounds that inhibit c-Met, their design, their synthesis, and their application as a pharmaceutical for the treatment of disease.

### THE NAMES OF THE PARTIES TO A JOINT RESEARCH AGREEMENT

None.

### REFERENCE TO SEQUENCE LISTING

None.

### BACKGROUND OF THE INVENTION

A tyrosine kinase is an enzyme that transfers a phosphate group from ATP to a tyrosine residue in a protein. Tyrosine kinases are a subgroup of the larger class of protein kinase. Fundamentally, a protein kinase is an enzyme that modifies a protein by chemically adding phosphate groups via phosphorylation. Such modification often results in a functional change to the target protein or substrate by changing the enzyme activity, cellular location or association with other proteins. Chemically, the kinase removes a phosphate group from ATP and covalently attaches it to one of three amino acids (serine, threonine or tyrosine) that have a free hydroxyl group. Most kinases act on both serine and threonine, and certain others, tyrosine. There are also a number of kinases that act on all three of these amino acids.

Tyrosine kinases are divided into two groups: cytoplasmic proteins and transmembrane receptor kinases. In humans, there are 32 cytoplasmic protein tyrosine kinases and 48 receptor-linked protein-tyrosine kinases.

c-Met is a receptor tyrosine kinase activated by Hepatocyte Growth Factor/Scatter Factor (HGF/SF). The c-met proto-oncogene, widely expressed in mammalian tissues, encodes a transmembrane receptor of 190-kDa heterodimer made of a 50-kDa extracellular a subunit linked by a disulfide bridge to a 145-kDa transmembranous catalytic β subunit. The hepatocyte growth factor (HGF), also called Scatter Factor (SF), is the ligand for the c-Met receptor. Seidel, C., Borset, M., Hjorth-Hansen, H., Sundan, Al., Waage, A., (1998) Role of Hepatocyte Growth Factor and Its Receptor c-Met in Multiple Myeloma Med Oncol 15, 145-53; Brset, M., Seidel, C., Hjorth-Hansen, H., Waage, A., Sundan, A. (1999) The Role of Hepatocyte Growth Factor and Its Receptor c-Met in Multiple Myeloma and Other Blood Malignancies Leukemia & Lymphoma 32, 249-256. Both HGF and c-Met are required for normal mammalian development, Christensen, J. G., Schreck, R., Burrows, J., Kuruganti, P., Chan, E., Le, P., Chen, J., Wang, X., Ruslim, L., Blake, R., Lipson, K. E., Ramphal, J., Do, S., Cui, J. J., Chemington, J. M., Mendel, D. B. (2003) A Selective Small Molecule Inhibitor of c-Met Kinase Inhibits c-Met-Dependent Phenotypes in Vitro and Exhibits Cytoreductive Antitumor Activity in Vivo Cancer Res 63, 7345-55, and are expressed predominantly in cells of epithelial and mesenchymal origin. DiRenzo, M. F., Narsimhan, R. P., Olivero, M., Bretti S., Giodano, S., Medico, E., Gaglia P., Zara P., Comoglio, P. M. (1991) Expression of the Met/HGF Receptor in Normal and Neoplastic Human Tissues Oncogene 6, 1997-2003; Sonnenberg, E., Weidner, K. M., Birchmeier, C. (1993) Expression of the Met-Receptor and Its Ligand, HGF-SF During Mouse Embryogenesis Exs 65, 381-94.

Generally, kinases are enzymes known to regulate the majority of cellular pathways, especially pathways involved in signal transduction or the transmission of signals within a cell. Because protein kinases have profound effect on a cell, kinase activity is highly regulated. Kinases can be turned on or off by phosphorylation (sometimes by the kinase itself -cis- phosphorylation/autophosphorylation) and by binding to activator proteins, inhibitor proteins or small molecules.

Deregulated kinase activity is a frequent cause of disease, particularly cancer where kinases regulate many aspect that control cell growth, movement and death. For example, neoplastic transformation in which multiple genetic defects such as translocation, mutations within oncogenes and the like, have been implicated in the development of leukemia. Many of these genetic defects have been identified as key components of signaling pathways responsible for proliferation and differentiation.

c-Met gene expression is induced in response to extracellular signals and also by its own ligand HGF/SF. HGF/SF expression in human glioblatoma cell lines down-regulates c-Met activated cellular events such as anchorage-independent cell growth and in vivo tumorigenicity and chemoresistance. Multiple signaling pathways have been associated with the biological responses mediated by c-Met activation. Abounader, R., et al. (2001) Signaling Pathways in the Induction of c-Met Receptor Expression by its Ligand Scatter Factor/Hepatocyte Growth Factor in Human Glioblastoma, J. Neurochem, 75, 1497-1508. When HGF/SF activates c-Met, the latter in turn may activate a number of kinase pathways, including, but not limited to, the pathway from Ras to Raf to Mek to the mitogen-activated protein kinase ERK1 to the transcription factor ETS1.

HGF/SF and c-Met are present in the developing and adult mammalian nervous system where they have a neurotrophic function, promote survival of motor neurons and are involved in the microglial reactions to central nervous system injuries. Both c-Met and HGF/SF genes are localized on human chromosome 7 that is linked to a number of human genes involved in tumor progression, invasion and metastasis. Id. Co-expression of unaltered c-Met and HGF/SF, as well as activating mutations, are oncogenic. Id.

c-Met plays a role in normal hematopoiesis, and is expressed in various lymphoid and leukemic cell lines. A wide variety of human tumors express both c-Met and HGF/SF and their expression contribute to the malignant progression of gliomas. In addition, overexpression of either HGF or c-Met is found in several cancers, and have been correlated with disease progression and clinical outcome. Ferracini, R., DiRenzo, M. F., Scotlandi, J., Baldini, N., Olivero, M., Lollini, P., Cremona, O., Campanacci, M., Comoglio, P. M. (1995) The Met/HGF Receptor Is Over-Expressed in Human Osteosarcomas and Is Activated By Either a Paracrine or an Autocrine Circuit Oncogene 10, 739-49; Rusciano, D., Lorenzoni, P., Burger, M. M. (1995) Expression of Constitutively Activated Hepatocyte Growth Factor/Scatter Factor Receptor (c-Met) in B16 Melanoma Cells Selected for Enhanced Liver Colonization Oncogene 11, 1979-87. Furthermore, the c-Met tyrosine kinase has been implemented in the development and progression of colon cancer, Herynk, M. H., Stoeltzing, O., Reinmuth, N., Parikh, N. U., Abounader, R., Laterra, J., Radinsky, R., Ellis, L. M., Gallick, G. E. (2003) Down-Regulation of c-Met Inhibits Growth in the Liver of Human Colorectal Carcinoma Cells Cancer Res 63, 2990-6, prostate cancer, Kim, S. J., Johnson, M., Koterba, K., Herynk, M. H., Uehara, H., Gallick, G. E. (2003) Reduced c-Met Expression By an Adenovirus Expressing a c-Met Ribozyme Inhibits Tumorigenic Growth and Lymph Node Metastases of PC3-LN4 Prostate Tumor Cells in an Orthotopic Nude Mouse Model Clin Cancer Res 9, 5161-70, and cancer in other organs, Longati, P., Comoglio, P. M., Bardelli, A. (2001) Receptor Tyrosine Kinases as Therapeutic Targets: the Model of the MET Oncogene Curr Drug Targets 2, 41-55, as well in blood malignancies such as multiple myeloma. Brset, M., Seidel, C., Hjorth-Hansen, H., Waage, A., Sundan, A. (1999) The Role of Hepatocyte Growth Factor and Its Receptor c-Met in Multiple Myeloma and Other Blood Malignancies Leukemia & Lymphoma 32, 249-256.

Thus, inhibiting c-Met has therapeutic value. For example, blockage with a single dose of one inhibitor, PHA-665752, has been shown to inhibit c-Met phosphorylation in tumor zenografts for to 12 hours. Christensen, J. G., Schreck, R., Burrows, J., Kuruganti, P., Chan, E., Le, P., Chen, J., Wang, X., Ruslim, L., Blake, R., Lipson, K. E., Ramphal, J., Do, S., Cui, J. J., Chemington, J. M., Mendel, D. B. (2003) A Selective Small Molecule Inhibitor of c-Met Kinase Inhibits c-Met-Dependent Phenotypes in Vitro and Exhibits Cytoreductive Antitumor Activity in Vivo Cancer Res 63, 7345-55. Therefore, a need exists for compounds useful in treating disease associated with regulation of the c-Met kinase and other tyrosine protein kinase related disorders.

### BRIEF SUMMARY OF THE INVENTION

Novel compounds and pharmaceutical compositions that inhibit c-Met have been found together with methods of structurally designing such compounds, methods of synthesizing and methods of using the compounds including methods for of inhibiting c-Met disorders in a patient by administering the compounds.

The present invention discloses a class of compounds useful in treating c-Met-mediated disorders and conditions, defined by the structural Formula I:

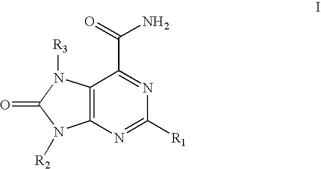

or salt, ester or prodrug thereof, wherein:
R₁ is independently selected from the group consisting of aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₁ is independently alkyl or cycloalkyl;
R₂ is aryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro; and
R₃ is independently selected from the group H, alkenyl, alkoxyalkyl, alkyl, alkynyl, cycloalkyl, cycloalkylalkyl, or haloalkyl;
provided that:
when R₂ is p-tolyl and R₃ is H, R₁ is not phenyl, 3,4-dimethoxyphenyl, 2-thienyl or cyclohexyl.

Except the present invention further discloses a class of compounds useful in treating c-Met-mediated disorders and conditions, defined by the structural Formula II:

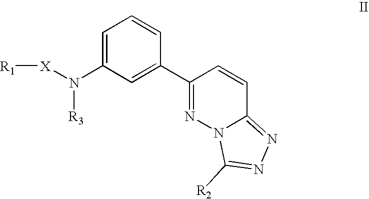

or salt, ester or prodrug, thereof, wherein:
R₁ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₂ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro;
R₃ is independently selected from the group H, alkenyl, alkoxyalkyl, alkyl, alkynyl, cycloalkyl, cycloalkylalkyl, or haloalkyl; and
X is independently selected from the group CO, SO2, SCO, or OCO;
provided that:
when R₁ is 5-(1,3-benzodioxole), 3,4,5-trimethoxyphenyl, 3-nitrophenyl or 3,4-dimethoxyphenyl and R₃ is H, and X is CO then R₂ is not H, 2-thienyl, 3-pyridyl, 4-pyridyl or methyl;
further provided that:
when R₁ is 2,5-dimethoxyphenyl, R₃ is H and X is SO2, R₂ is not H or 2-thienyl.

Compounds according to the present invention possess useful c-Met inhibiting or modulating activity and may be used in the treatment or prophylaxis of a disease or condition in which c-Met plays an active role. Thus, in the broad aspect, the present invention provides for pharmaceutical compositions comprising one or more compounds of the present invention together with a pharmaceutically acceptable carrier as well as methods of making and using the compounds and compositions. The present invention provides methods for treating a c-Met-mediated disorder in a patient in need of such treatment comprising administering to said patient a therapeutically effective amount of a compound or composition according to the subject invention. The present invention also contemplates the use of compounds disclosed herein for use in the manufacture of a medicament for the treatment of a diseases or condition ameliorated by the inhibition of c-Met.

### BRIEF DESCRIPTION OF THE SEVERAL VIEWS OF THE DRAWINGS

FIG. 1 is a schematic view of c-Met.

FIG. 2 is a flow chart showing the compound selection strategy used in connection with the present invention.

### DETAILED DESCRIPTION OF THE INVENTION

In certain embodiments, the compounds of the present invention have structural Formula I

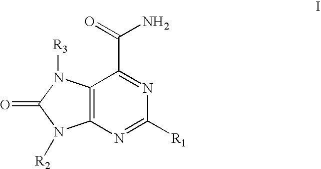

or salt, ester or prodrug thereof, wherein:
R₁ is independently selected from the group consisting of aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₁ is independently alkyl or cycloalkyl;
R₂ is aryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro; and
R₃ is independently selected from the group H, alkenyl, alkoxyalkyl, alkyl, alkynyl, cycloalkyl, cycloalkylalkyl, or haloalkyl;
provided that:
when R₂ is p-tolyl and R₃ is H, R₁ is not phenyl, 3,4-dimethoxyphenyl, 2-thienyl or cyclohexyl.

In certain other embodiments, the compounds of the present invention have structural Formula II:

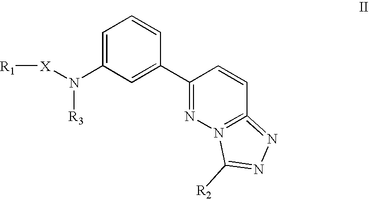

or salt, ester or prodrug, thereof, wherein:
R₁ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₂ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro;
R₃ is independently selected from the group H, alkenyl, alkoxyalkyl, alkyl, alkynyl, cycloalkyl, cycloalkylalkyl, or haloalkyl; and
X is independently selected from the group CO, SO2, SCO, or OCO;
provided that:
when R₁ is 5-(1,3-benzodioxole), 3,4,5-trimethoxyphenyl, 3-nitrophenyl or 3,4-dimethoxyphenyl and R₃ is H, and X is CO then R₂ is not H, 2-thienyl, 3-pyridyl, 4-pyridyl or methyl;
further provided that:
when R₁ is 2,5-dimethoxyphenyl, R₃ is H and X is SO2, R₂ is not H or 2-thienyl.

Specific compounds of particular interest consist of compounds and pharmaceutically-acceptable salts, esters and prodrugs thereof as follows:
9-(4-methylphenyl)-8-oxo-2-(2-thienyl)-8,9-dihydro-7H-purine-6-carboxamide;
2-(1H-imidazol-5-yl)-9-(4-methylphenyl)-8-oxo-8,9-dihydro-7H-purine-6-carboxamide;
2-(1H-indol-5-yl)-9-(4-methylphenyl)-8-oxo-8,9-dihydro-7H-purine-6-carboxamide;
2-(2,4-dinitrophenyl)-9-(4-methylphenyl)-8-oxo-8,9-dihydro-7H-purine-6-carboxamide;
9-(4-methylphenyl)-8-oxo-2-(4-phenyl-2-thienyl)-8,9-dihydro-7H-purine-6-carboxamide;
2-(2,2′-bithien-5-yl)-9-(4-methylphenyl)-8-oxo-8,9-dihydro-7H-purine-6-carboxamide;
2-(1-benzothien-3-yl)-9-(4-methylphenyl)-8-oxo-8,9-dihydro-7H-purine-6-carboxamide;
2-(1H-imidazol-2-yl)-9-(4-methylphenyl)-8-oxo-8,9-dihydro-7H-purine-6-carboxamide;
2-(4-chloro-2,6-difluorophenyl)-9-(4-methylphenyl)-8-oxo-8,9-dihydro-7H-purine-6-carboxamide;
2-(3,5-dihydroxyphenyl)-9-(4-methylphenyl)-8-oxo-8,9-dihydro-7H-purine-6-carboxamide;
2-(6-bromopyridin-2-yl)-9-(4-methylphenyl)-8-oxo-8,9-dihydro-7H-purine-6-carboxamide;
4-[6-(aminocarbonyl)-9-(4-methylphenyl)-8-oxo-8,9-dihydro-7H-purin-2-yl]-2,6-dimethoxyphenyl acetate;
2-(2-chloro-6-nitrophenyl)-9-(4-methylphenyl)-8-oxo-8,9-dihydro-7H-purine-6-carboxamide;
2-(2-fluoropyridin-3-yl)-9-(4-methylphenyl)-8-oxo-8,9-dihydro-7H-purine-6-carboxamide;
2-(3,4-dimethoxyphenyl)-9-(4-methylphenyl)-8-oxo-8,9-dihydro-7H-purine-6-carboxamide;
2-(4-chloro-3-nitrophenyl)-9-(4-methylphenyl)-8-oxo-8,9-dihydro-7H-purine-6-carboxamide;
2-[3-fluoro-5-(trifluoromethyl)phenyl]-9-(4-methylphenyl)-8-oxo-8,9-dihydro-7H-purine-6-carboxamide;
2-(3-formylphenyl)-9-(4-methylphenyl)-8-oxo-8,9-dihydro-7H-purine-6-carboxamide;
9-(4-methylphenyl)-8-oxo-2-phenyl-8,9-dihydro-7H-purine-6-carboxamide;
2-cyclohexyl-9-(4-methylphenyl)-8-oxo-8,9-dihydro-7H-purine-6-carboxamide;
N-[3-(3-thien-2-yl[1,2,4]triazolo[4,3-b]pyridazin-6-yl)phenyl]-1,3-benzodioxole-5-carboxamide;
3,4,5-trimethoxy-N-[3-(3-phenyl[1,2,4]triazolo[4,3-b]pyridazin-6-yl)phenyl]benzamide;
3,4,5-trimethoxy-N-(3-[1,2,4]triazolo[4,3-b]pyridazin-6-ylphenyl)benzamide;
3,4,5-trimethoxy-N-[3-(3-thien-2-yl[1,2,4]triazolo[4,3-b]pyridazin-6-yl)phenyl]benzamide;
3,4,5-trimethoxy-N-[3-(3-pyridin-3-yl[1,2,4]triazolo[4,3-b]pyridazin-6-yl)phenyl]benzamide;
3,4,5-trimethoxy-N-{3-[3-(1H-pyrrol-2-yl)[1,2,4]triazolo[4,3-b]pyridazin-6-yl]phenyl}benzamide;
3-nitro-N-[3-(3-phenyl[1,2,4]triazolo[4,3-b]pyridazin-6-yl)phenyl]benzamide;
3-nitro-N-(3-[1,2,4]triazolo[4,3-b]pyridazin-6-ylphenyl)benzamide;
3-nitro-N-[3-(3-thien-2-yl[1,2,4]triazolo[4,3-b]pyridazin-6-yl)phenyl]benzamide;
3-nitro-N-{3-[3-(1H-pyrrol-2-yl)[1,2,4]triazolo[4,3-b]pyridazin-6-yl]phenyl}benzamide;
3,4-dimethoxy-N-[3-(3-phenyl[1,2,4]triazolo[4,3-b]pyridazin-6-yl)phenyl]benzamide;
3,4-dimethoxy-N-[3-(3-thien-2-yl[1,2,4]triazolo[4,3-b]pyridazin-6-yl)phenyl]benzamide;
3,4-dimethoxy-N-[3-(3-pyridin-3-yl[1,2,4]triazolo[4,3-b]pyridazin-6-yl)phenyl]benzamide;
3,4-dimethoxy-N-[3-(3-pyridin-4-yl[1,2,4]triazolo[4,3-b]pyridazin-6-yl)phenyl]benzamide;
3,4-dimethoxy-N-{3-[3-(1H-pyrrol-2-yl)[1,2,4]triazolo[4,3-b]pyridazin-6-yl]phenyl}benzamide;
3,4-dimethoxy-N-[3-(3-methyl[1,2,4]triazolo[4,3-b]pyridazin-6-yl)phenyl]benzamide;
2,5-dimethoxy-N-(3-[1,2,4]-triazolo[4,3-b]pyridazin-6-ylphenyl)benzenesulfonamide;
2,5-dimethoxy-N-[3-(3-thien-2-yl[1,2,4]triazolo[4,3-b]pyridazin-6-yl)phenyl]benzenesulfonamide; and
S-phenyl 3-[3-(2-furyl)[1,2,4]triazolo[4,3-b]pyridazin-6-yl]phenylthiocarbamate.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(CH═CH—), (—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. In certain embodiments, alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-diethylamino and the like.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted as defined herein with one, two, three, four, or five substituents.

The term “aminoalkyl,” as used herein, alone or in combination, refers to an amino group attached to the parent molecular moiety through an alkyl group. Examples include aminomethyl, aminoethyl and aminobutyl.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an C(NH)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄═ derived from benzene. Examples include benzothiophene and benzimidazole.

The term “O-carbamyl” as used herein, alone or in combination, refers to a —OC(O)NRR′, group— with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.

The term “ester,” as used herein, alone or in combination, refers to a carboxyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.] etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.] and isothiazolyl; unsaturated condensed heterocyclic groups containing 1 to 2 sulfur atoms and 1 to 3 nitrogen atoms [e.g., benzothiazolyl, benzothiadiazolyl, etc.] and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkylalkenyl,” as used herein, alone or in combination, refers to a heterocycle group attached to the parent molecular moiety through an alkenyl group.

The term “heterocycloalkylalkoxy,” as used herein, alone or in combination, refers to a heterocycle group attached to the parent molecular group through an oxygen atom.

The term “heterocycloalkylalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′ SR— group, where R and R′ are as defined herein.

The term “mercaptomercaptyl” as used herein, alone or in combination, refers to a RSR′S group, where R and R′ are as defined herein.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —SO₂.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanate” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

c-Met is a trysoine kinase distinguishable from most other such proteins on the basis of its biosynthesis and its structural features. Met is synthesized as a single-chain precursor which under goes intracellular proteolytic cleavage, yielding a disulfide-linked heterodimer. The C-terminal (intracellular region) of Met contains a multifunctional docking site that binds to various signaling molecules. These features define a Met receptor kinase family consisting of three related proteins, Met, Ron and c-Sea.

The c-Met protein is often designated as c-Met in the literature together with a wide variety of other possible variations, including but not limited to, c met, cmet, c-met, Met, Met, MET, HGF/Met receptor, Met receptor, and the HGF receptor. Likewise, the gene encoding c-Met is often designated in the literature as Met, c-Met, and MET protooncogene. Moreover, as with protein designations, the terms c-Met, c-met, c-MET, MET, MET, and c-Met can be associated with the gene that encodes the protein and variations thereof. Therefore, as used herein, any one of a number of possible variation of the terms designating the c-Met protein and the gene encoding the protein can and may be used interchangeably herein.

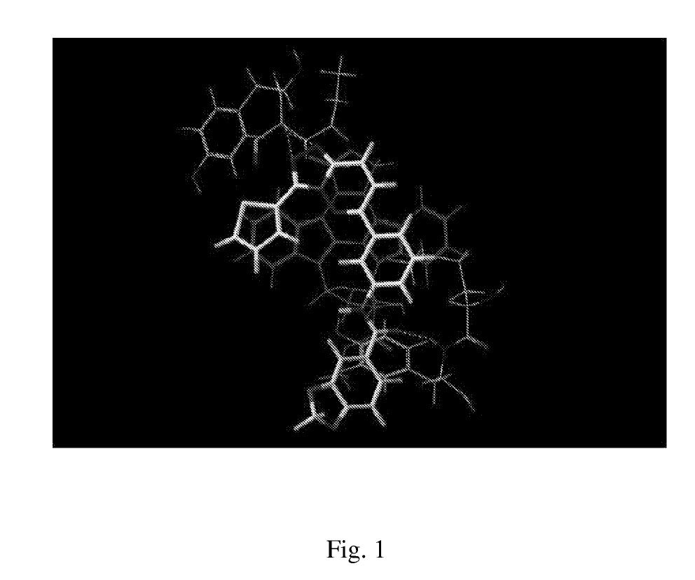

c-Met is a type of receptor tyrosine kinase (RTK) involved in signal transduction as shown in the schematic of FIG. 1. In general, RTKs are monomeric surface receptors that dimerize upon activation. RTKs have an extracellular binding domain, a transmembrane domain, and an intracellular kinase domain. Ligand binding to the extracellular domain induces dimerization of the surface receptor which in turn induces phosphorylation of tyrosine residues within an “activation loop” of the intracellular kinase domain.

As noted above, the ligand of the Met receptor is hepatocyte growth factor (HGF), also known as scatter factor (SF). HGF/SF is a multifunctional factor that affects different cells including epithelium, endothelium, myoblasts, spinal motor neurons and hematopoietic cells. Activation of the Met receptors occurs through a multistep process including (a) HGF-induced dimerization, (b) receptor phosphorylation on tyrosine residues, and (c) recruitment and activation of cytoplasmic signaling molecules. Signaling pathways activated by the HGF-Met interaction mediate cell adhesion and motility.

In addition to regulating normal cell functions, Met is involved in malignant cell transformation. Increased Met expression has been found in papillary carcinomas of the thyroid gland, in carcinomas of colon, pancreas and ovary, in osteogenic sarcomas and in other types of cancer. Point mutations in MET have been identified in hereditary and sporadic papillary renal carcinomas, hepatocellular and gastric carcinomas and head and neck squamous carcinomas. The role of HGF and Met has been indicated in tumor invasive growth. Met overexpression and hyperactivation are reported to correlate with metastatic ability of tumor cells.

HGF binding to c-Met results in receptor autophosphorylation and upregulation of Met kinase activity and stimulates a number of intracellular pathways. In normal cells, Met activation is a ligand-dependent transient event. In tumor cells, Met activity is often constitutively upregulated. Met can also be activated in an HGF-independent manner in tumors, particularly as a result of overexpression of Met. Furthermore, mutation in the MET gene can lead to active, typically ligand-independent, Met signally in tumor cells.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002).

Thus, preferred salts include hydrochloride, hydrobromide, sulfonate, citrate, tartrate, phosphonate, lactate, pyruvate, acetate, succinate, oxalate, fumarate, malate, oxaloacetate, methanesulfonate, ethanesulfonate, p-toluenesulfonate, benzenesulfonate and isethionate salts of compounds of the present invention. A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as siliceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating c-Met-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of c-Met-mediated disorders.

The compounds of the subject invention may be useful for the treatment or disorders of a wide variety of condition where inhibition or modulation of c-Met is useful. Disorders or conditions advantageously treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods can also be used to treat the fibrosis which occurs with radiation therapy. The present compounds and methods can be used to treat subjects having adenomatous polyps, including those with familial adenomatous polyposis (FAP). Additionally, the present compounds and methods can be used to prevent polyps from forming in patients at risk of FAP.

Specific treatable neoplasms include systemic mast cell disorders, seminoma, acute myelogenous leukemia (AML), gastrointestinal stromal tumors (GISTs) or hypopigmentary disorders.

A c-Met condition or disorder can be the result of a regulatory-type mutation. Protein residues may be mutated in a membrane domain or a juxtamembrane domain that normally inhibits ligand-independent kinase activation. In addition, c-Met disorder may be due to over-expression, inappropriate timing of activation, or by inappropriate levels or activity of ligands that bind to the kinase receptor.

The compounds of the such invention may be also used in as combination therapy together with existing tyrosine kinase inhibitors such as dasatinib (BMS-354825, Bristol Myers Squibb; a small-molecule, ATP-competitive inhibitor of SRC and ABL tyrosine kinases having a potency in the low nanomolar range), imatinib mesylate (a.k.a. IMATINIB®, STI-571, GLEEVEC® or GLIVEC®, Novartis), gefitinib (IRESSA®, Astra Zeneca), erlotinib (TARCEVA®, OSI Pharmaceuticals), AMN107 (Novartis), and sunitinib malate (a.k.a. SU11248, SUTENT®, Pfizer). c-Met mutations known to be sensitive to IMATINIB®, include Val560Gly, Glu839Lys, and Asp820Gly. c-Met mutations known to be resistant to IMATINIB®, include Asp816Val/Phe/Tyr.

Other disorders or conditions which can be advantageously treated by the compounds of the present invention are inflammation. The compounds of the present invention are useful as anti-inflammatory agents with the additional benefit of having significantly less harmful side effects. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as inflammatory bowel disease, Crohn's disease, gastritis, irritable bowel syndrome and ulcerative colitis. The compounds may also be used in the treatment of pulmonary inflammation, such as that associated with cystic fibrosis. In addition, compounds of invention are also useful in organ transplant patients either alone or in combination with conventional immunomodulators.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered orally or by injection (intravenous or subcutaneous). The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

The design of c-Met inhibitors provided herein includes the selection of screening compounds, in-silico screening via docking, analysis of docking results, preliminary selection of compounds with a degree of specificity towards c-Met, and final selection of candidate compounds.

Compounds from three vendors: Asinex (Winston-Salem, N.C.), BioFocus (Saffron Walden, Essex, UK), and LifeChem (Burlington, ON, Canada) were screened in-silico against c-Met and c-Kit via docking the individual ligands into the receptor binding site.

The traditional metric for this type of screening has been the individual docking scores, which aim to capture the binding affinity of the ligand and receptor. Though useful in ranking ligands, the scoring methods are imperfect, so the consensus of several scoring methods (Clark, et al., (2002) J Mol Graph Model 20, 281-95) is generally utilized in the final selection. In the present study, the consensus scoring was completed with the scoring methods provided in the FlexX (Kramer, et al., (1999) Proteins 37, 228-41) module within Sybyl 7.1 (Tripos, Inc., St. Louis, Mo.). Over 32,000 compounds were screened in this manner. While compounds with the highest scores were of interest, additional metrics were also considered. For example, an emphasis was placed on identifying those compounds with a larger difference in scores when compared to other kinases. The underlying principle is that selections based on the larger score differences should translate into greater selectivity towards one kinase.

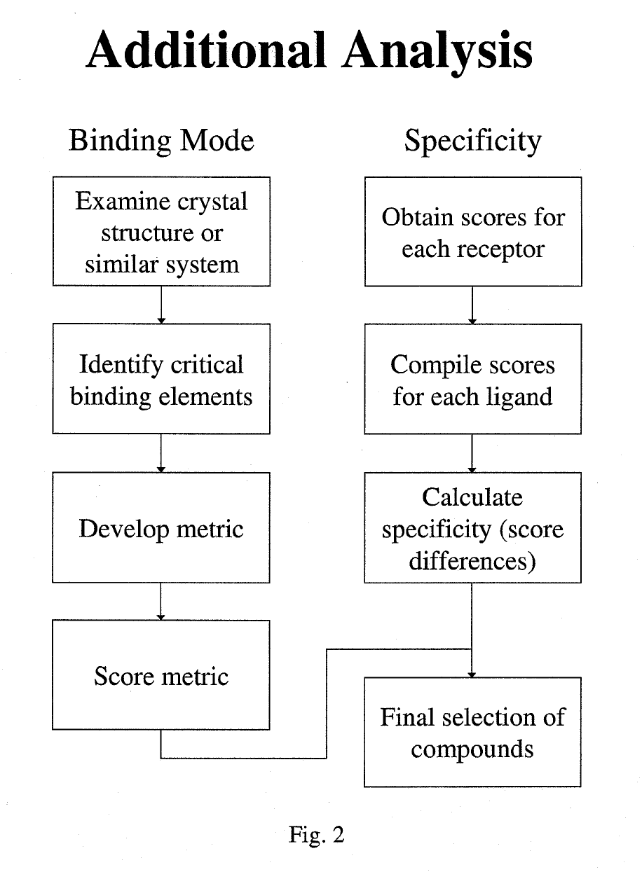

For the present study, the selectivity was compared in-silico against c-Kit, a player in Leukemia, gastrointestinal stromal tumors (GIST), and other disorders. Another element used in the selection of compounds was the evaluation of binding mode. Utilization of specific interactions along with the docking scores has been shown to increase the hit ratio of in-silico screening (Hindle, et al., (2002) J Comput Aided Mol Des 16, 129-49; Boehm, et al., (2002) Reviews in Computational Chemistry 18, 41-87). Information on these binding interactions was derived from an inhibitor bound crystal structure from Mol, et al. ((2004) Journal of Biological Chemistry 279:30, 31655-31663). An example of such a structure is provided by FIG. 1 which is a schematic showing one of top scoring compounds bound to the ATP binding pocket of c-Met. This level of analysis was not available as part of the normal docking interface, so code was written to analyze the data and provide consensus information. A flowchart showing this strategy is shown in FIG. 2. The analysis included identifying hydrogen bonding elements within each docking configuration for each ligand and comparing those against the specific areas of the receptor site. Only interactions with Met170 and Tyr1230 were mandated and those compounds within a particular distance were flagged and the combination of elements was used as a filter for the binding mode. This procedure was followed for docked configurations determined for each of the compounds in the screening libraries. Several candidate compounds were identified in this manner. After restricting to a particular binding mode, the above mentioned filters were then applied in the order of docking score, consensus score and, lastly, for selectivity. This computational strategy indicated that compounds based on a 1,2,4-Triazolo[4,3-b]pyridazine and 8H-Purin-8-one, 7,9-dihydro- cores may be effective for inhibition of c-Met kinase.

### General Synthetic Methods for Preparing Compounds

The following scheme can be used to practice the present invention.

Examples 1-39 can be synthesized using one of the following general synthetic procedures set forth in Scheme 1, Scheme 2 or Scheme 3.

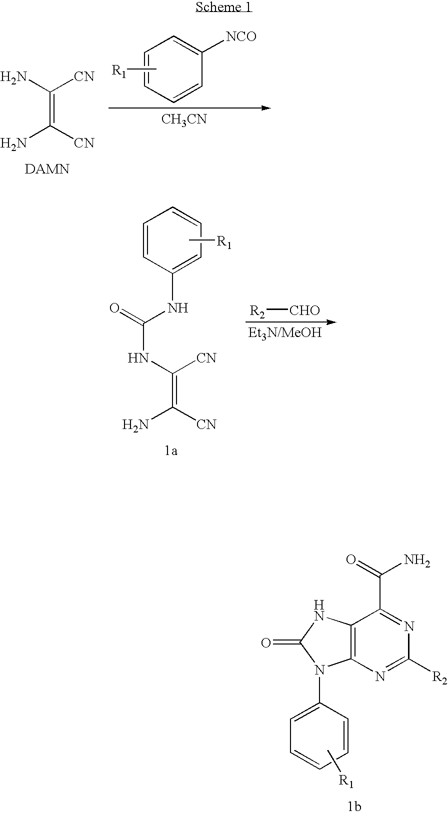

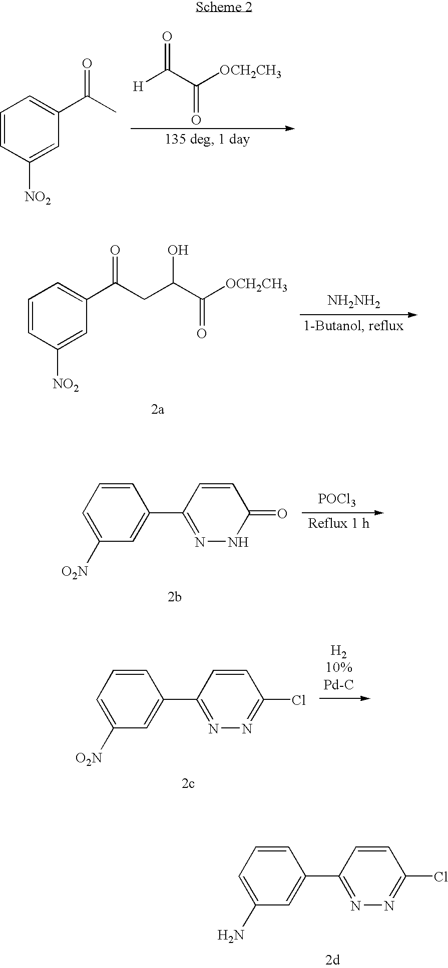

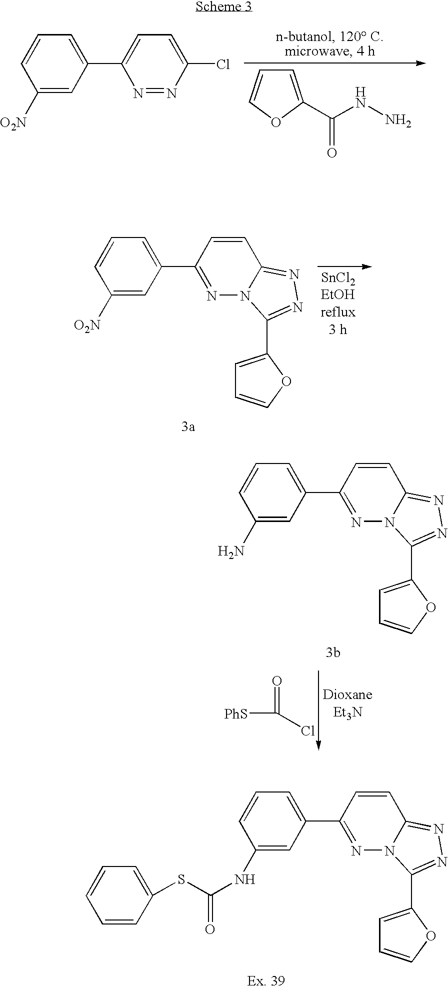

All chemicals and solvents were obtained from Sigma-Aldrich (Milwaukee, Wis.) or Fisher Scientific (Pittsburgh, Pa.) and used without further purification. ¹H-NMR and ¹³C-NMR spectra were recorded on an IBM-Brucker Avance 300 (300 MHz for ¹H-NMR and 75.48 MHz for ¹³C-NMR), and IBM-Brucker Avance 500 (500 MHz for ¹H-NMR and 125.76 MHz for ¹³C-NMR), spectrometers. Chemical shifts (8) are determined relative to CDCl₃ (referenced to 7.27 ppm (δ) for ¹H-NMR and 77.0 ppm for ¹³C-NMR) or DMSO-d₆ (referenced to 2.49 ppm (δ) for ¹H-NMR and 39.5 ppm for ¹³C-NMR). Proton-proton coupling constants (J) are given in Hertz and spectral splitting patterns are designated as singlet (s), doublet (d), triplet (t), quadruplet (q), multiplet or overlapped (m), and broad (br). Low resolution mass spectra (ionspray, a variation of electrospray) were acquired on a Perkin-Elmer Sciex API 100 spectrometer or Applied Biosystems Q-trap 2000 LC-MS-MS. Flash chromatography was performed using Merk silica gel 60 (mesh size 230-400 ASTM) or using an Isco (Lincon, Nebr.) combiFlash Companion or SQ16x flash chromatography system with RediSep columns (normal phase silica gel (mesh size 230-400ASTM) and Fisher Optima™ grade solvents. Thin-layer chromatography (TLC) was performed on E. Merk (Darmstadt, Germany) silica gel F-254 aluminum-backed plates with visualization under UV (254 nm) and by staining with potassium permanganate or ceric ammonium molybdate.

Illustrative of Scheme 1 is the synthesis of Examples 1-20.

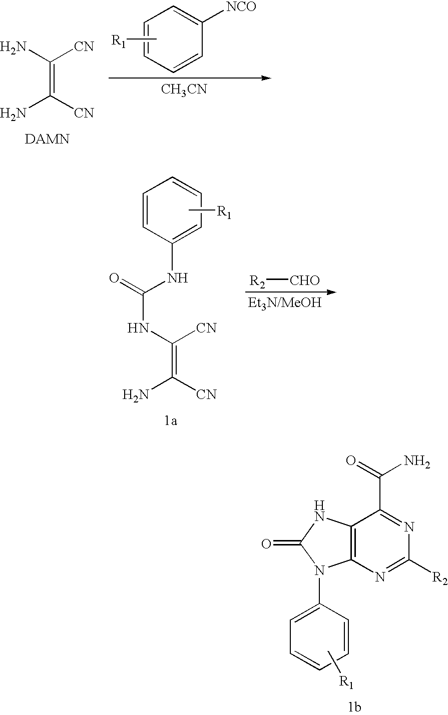

The target purinone carboxamides may be prepared by acylation of diaminomaleonitrile with an isocyanate (RNCO) to afford the corresponding urea, which is then condensed with an aldehyde (R₁CHO) to afford the requisite purinone carboxamide.

### General Procedure:

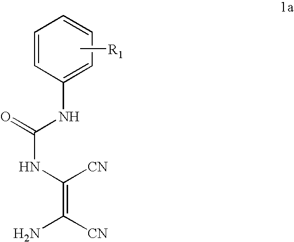

A solution of DAMN (10 g, 92.5 mmol) in dry acetonitrile (150 mL) was kept stirring in an ice bath, under a nitrogen atmosphere, in a round-bottom flask equipped with a serum cap. Substituted phenyl isocyanate (98 mmol) was added dropwise with a syringe through the serum cap. The mixture was stirred at room temperature for 24 h and the solid was filtered and washed with acetonitrile and diethyl ether to yield the intermediate 1a.

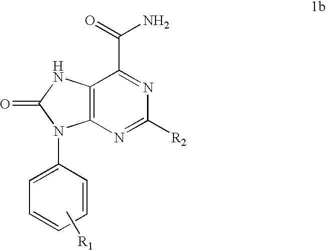

Triethylamine (0.43 mL) was added to a suspension of diaminomaleonitrile urea 1a (0.44 mmol) and aldehyde (0.97 mmol) in methanol (15 mL). The solution was stirred at room temperature overnight. The precipitate formed was filtered and washed with methanol and diethyl ether to give the compound 1b.

The invention is further illustrated by the following examples.

### Example 1

### 9-(4-methylphenyl)-8-oxo-2-(2-thienyl)-8,9-dihydro-7H-purine-6-carboxamide

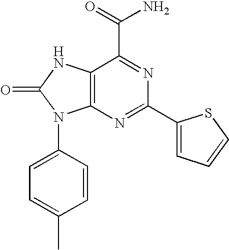

IR: 3429.6, 3217.1, 1718.6, 1675.4, 1598.6, 1581.7, 1535.9, 1517.1, 1469.3, 1436.6, 1391.1, 1338.9, 1221.3, 1174.9, 1118.9, 1005.9, 851.6, 813.4, 704.0, 680.8 cm⁻¹; ¹H NMR (600 MHz, DMSO-d₆) δ 2.39 (s, 3H), 7.15 (t, 1H, J=4.2 Hz), 7.37 (dd, 2H, J=4.8 Hz), 7.64 (d, 2H, J=4.8 Hz), 7.62 (s, 1H), 7.98 (s, 1H), 8.04 (d, 1H, J=2.4 Hz), 8.28 (s, 1H), 11.74 (s, 1H); ¹³C NMR (150 MHz, DMSO-d₆) δ 20.82, 119.44, 126.18, 128.29, 128.47, 129.41, 130.01, 132.61, 137.50, 142.53, 151.88, 152.74, 152.89, 165.35; MS (ESI-negative ion) m/z (relative intensity) 307.3 (32), 350.3 (100).

### Example 2

### 2-(1H-imidazol-5-yl)-9-(4-methylphenyl)-8-oxo-8,9-dihydro-7H-purine-6-carboxamide

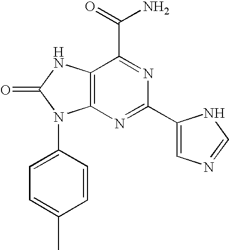

IR: 3170.8, 1743.3, 1684.4, 1595.8, 1575.4, 1518.6, 1473.0, 1414.3, 1364.9, 1122.6, 1002.7, 872.8, 823.2, 807.0, 707.9686.8 cm⁻¹; ¹H NMR (600 MHz, DMSO-d₆) δ 2.39 (s, 3H), 7.38 (d, 2H, J=7.8 Hz), 7.45 (brs, 1H), 7.53 (d, 2H, J=7.8 Hz), 7.85 (s, 1H), 7.97 (s, 1H), 8.69 (brs, 1H), 11.71 (brs, 1H), 12.80 (brs, 1H); ¹³C NMR (150 MHz, DMSO-d₆) δ 20.80, 126.38, 129.43, 130.05, 137.49, 152.93, 153.05, 165.51; MS (ESI-negative ion) m/z (relative intensity) 334.1 (100), 669.4 (12).

### Example 3

### 2-(1H-indol-5-yl)-9-(4-methylphenyl)-8-oxo-8,9-dihydro-7H-purine-6-carboxamide

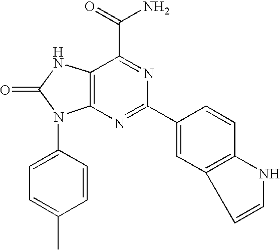

IR: 3441.8, 3292.1, 3240.8, 2981.7, 1720.1, 1671.8, 1599.1, 1580.7, 1518.7, 1455.3, 1389.3, 1227.0, 1170.4, 1033.1, 878.5, 887.8, 810.2, 760.4, 718.6 cm⁻¹; ¹H NMR (600 MHz, DMSO-d₆) δ 2.43 (s, 3H), 6.51 (s, 1H), 7.37 (s, 1H), 7.41 (s, 1H), 7.42 (d, 2H, J=7.2 Hz), 7.60 (d, 2H, J=7.2 Hz), 7.96 (s, Hz), 8.22 (d, 1H, J=9.0 Hz), 8.47 (s, 1H), 8.69 (s, 1H), 11.21 (s, 1H), 11.61 (s, 1H); ¹³C NMR (150 MHz, DMSO-d₆) δ 20.85, 102.19, 111.13, 120.31, 121.25, 126.35, 127.74, 128.11, 129.49, 130.29, 133.01, 137.26, 137.38, 152.94, 156.49, 165.91; MS (ESI-negative ion) m/z (relative intensity) 383.4 (100), 767.9 (13).

### Example 4

### 2-(2,4-dinitrophenyl)-9-(4-methylphenyl)-8-oxo-8,9-dihydro-7H-purine-6-carboxamide

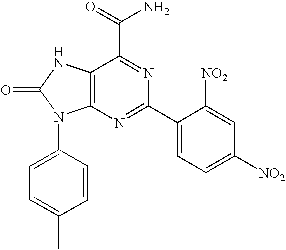

IR: 3504.5, 3383.0, 3102.2, 1743.7, 1682.5, 1572.2, 1524.2, 1461.2, 1343.6, 898.6, 815.0, 751.6, 735.9 cm⁻¹; ¹H NMR (600 MHz, DMSO-d₆) δ 2.40 (s, 3H), 7.36 (d, 2H, J=7.2 Hz), 7.50 (d, 2H, J=7.2 Hz), 8.05 (s, 1H), 8.08 (s, 1H), 8.54 (d, 1H, J=8.4 Hz), 8.60 (d, 1H, J=8.4 Hz), 8.75 (s, 1H), 12.05 (brs, 1H); ¹³C NMR (150 MHz, DMSO-d₆) δ 21.99, 120.64, 126.93, 127.53, 130.57, 130.81, 133.76, 134.06, 136.45, 138.74, 149.02, 150.40, 153.86, 166.12; MS (ESI-negative ion) m/z (relative intensity) 391.4 (21), 434.3 (100).

### Example 5

### 9-(4-methylphenyl)-8-oxo-2-(4-phenyl-2-thienyl)-8,9-dihydro-7H-purine-6-carboxamide

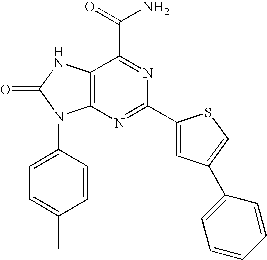

IR: 3199.2, 1731.8, 1670.2, 1597.1, 1518.8, 1472.9, 1442.9, 1388.1, 1170.7, 1048.4, 1022.2, 1006.0, 878.8, 814.6, 766.6, 738.5, 687.3 cm⁻¹; ¹H NMR (600 MHz, DMSO-d₆) δ 2.54 (s, 3H), 7.33 (t, 1H, J=8.4 Hz), 7.40 (d, 2H, J=7.8 Hz), 7.45 (t, 2H, J=8.4 Hz), 7.56 (d, 2H, J=7.8 Hz), 7.81 (d, 2H, J=7.8 Hz), 7.98 (s, 1H), 8.04 (s, 1H), 8.52 (s, 1H), 8.67 (s, 1H), 11.78 (s, 1H); ¹³C NMR (150 MHz, DMSO-d₆) δ 20.82, 119.52, 124.01, 125.94, 126.17, 126.98, 127.40, 128.92, 129.42, 130.01, 132.56, 134.84, 137.51, 142.13, 143.14, 151.59, 152.75, 152.87, 165.42; MS (ESI-negative ion) m/z (relative intensity) 383.4 (11), 426.3 (55.6).

### Example 6

### 2-(2,2′-bithien-5-yl)-9-(4-methylphenyl)-8-oxo-8,9-dihydro-7H-purine-6-carboxamide

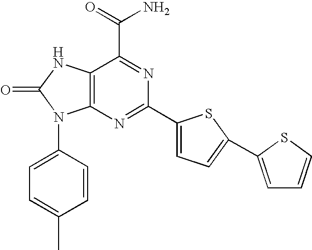

IR: 3435.8, 3217.3, 1732.8, 1678.2, 1597.3, 1518.2, 1474.4, 1458.2, 1393.3, 1229.9, 1174.9, 1049.7, 1004.9, 872.8, 803.2, 763.7, 682.8 cm⁻¹; ¹H NMR (600 MHz, DMSO-d₆) δ 2.42 (s, 3H), 7.12 (m, 1H), 7.34 (d, 1H, J=3.6 Hz), 7.40 (s, 1H), 7.42 (m, 2H), 7.54 (s, 1H), 7.56 (m, 2H), 7.97 (s, 1H), 7.99 (d, 1H, J=3.6 Hz), 8.32 (s, 1H), 11.75 (s, 1H); ¹³C NMR (150 MHz, DMSO-d₆) δ 20.93, 119.59, 124.81, 124.97, 126.21, 126.39, 128.64, 129.58, 130.07, 132.74, 136.46, 137.68, 139.43, 140.98, 151.52, 152.86, 153.00, 165.39; MS (ESI-negative ion) m/z (relative intensity).

### Example 7

### 2-(1-benzothien-3-yl)-9-(4-methylphenyl)-8-oxo-8,9-dihydro-7H-purine-6-carboxamide

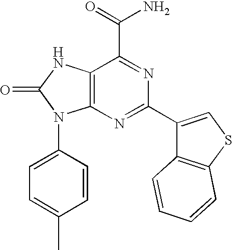

IR: 3444.2, 3180.1, 2922.1, 1740.0, 1681.5, 1599.2, 1516.2, 1457.3, 1388.71, 1159.7, 1004.0, 874.5, 811.0, 763.7, 722.5, 704.7, 667.3 cm⁻¹; ¹H NMR (600 MHz, DMSO-d₆) δ 2.44 (s, 3H), 7.41 (m, 2H), 7.44 (d, 2H, J=7.8 Hz), 7.66 (d, 2H, J=7.8 Hz), 8.02 (s, 1H), 8.05 (m, 1H), 8.42 (s, 1H), 8.91 (s, 1H), 8.97 (s, 1H), 11.77 (s, 1H); ¹³C NMR (150 MHz, DMSO-d₆) δ 20.86, 123.02, 124.51, 124.82, 125.23, 126.12, 129.37, 130.17, 131.78, 133.09, 133.51, 136.42, 137.48, 140.43, 152.49, 152.79, 165.64; MS (ESI-negative ion) m/z (relative intensity) 400.2 (100), 357.1 (24).

### Example 8

### 2-(1H-imidazol-2-yl)-9-(4-methylphenyl)-8-oxo-8,9-dihydro-7H-purine-6-carboxamide

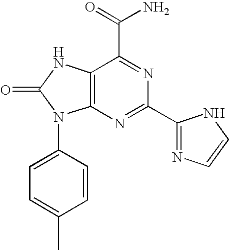

IR: 3178.6, 1736.5, 1681.4, 1593.8, 1580.7, 1518.7, 1455.5, 1376.3, 1005.3, 888.81, 721.1 cm⁻¹; ¹H NMR (600 MHz, DMSO-d₆) δ 2.41 (s, 3H), 7.04 (brs, 1H), 7.39 (d, 2H, J=7.8 Hz), 7.50 (d, 2H, J=7.8 Hz), 7.98 (s, 1H), 8.78 (s, 1H), 11.78 (s, 1H), 12.87 (brs, 1H); ¹³C NMR (150 MHz, DMSO-d₆) δ 20.91, 119.80, 126.97, 129.61, 130.11, 132.06, 137.91, 144.49, 148.32, 153.16, 153.58, 165.51; MS (ESI-negative ion) m/z (relative intensity) 334.1 (100), 669.4 (10), 291.0 (17).

### Example 9

### 2-(4-chloro-2,6-difluorophenyl)-9-(4-methylphenyl)-8-oxo-8,9-dihydro-7H-purine-6-carboxamide

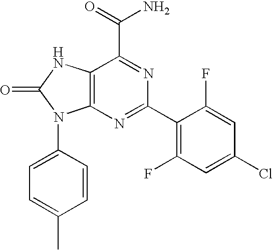

IR: 3501.4, 3383.1, 3178.8, 2923.7, 1753.3, 1690.2, 1595.2, 1566.7, 1518.5, 1465.4, 1417.6, 1388.4, 1191.2, 1167.9, 1132.9, 1008.0, 875.3, 816.6, 807.2, 666.70, cm⁻¹; ¹H NMR (600 MHz, DMSO-d₆) δ 2.39 (s, 3H), 7.31 (m, 1H), 7.35 (d, 2H, J=7.8 Hz), 7.48 (d, 2H, J=7.8 Hz), 7.77 (m, 1H), 7.94 (s, 1H), 8.11 (s, 1H), 11.93 (s, 1H); ¹³C NMR (150 MHz, DMSO-d₆) δ 20.71, 113.16, 113.29, 115.85, 115.97, 126.42, 129.46, 129.68, 131.27, 131.34, 132.68, 137.80, 147.33, 152.93, 154.25, 155.91, 164.97; MS (ESI-negative ion) m/z (relative intensity) 414.1 (100), 370.5 (22).

### Example 10

### 2-(3,5-dihydroxyphenyl)-9-(4-methylphenyl)-8-oxo-8,9-dihydro-7H-purine-6-carboxamide

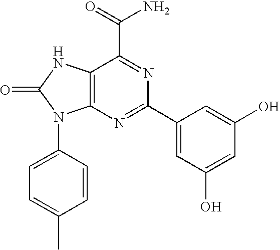

IR: 3430.07, 3227.98, 1731.5, 1677.9, 1598.2, 1517.8, 1471.6, 1416.0, 1392.3, 1266.7, 1228.7, 1175.9, 1050.8, 1033.1, 1004.3, 873.0, 812.4, 762.1, 693.4 cm⁻¹; ¹H NMR (600 MHz, DMSO-d₆) δ 2.42 (s, 3H), 6.30 (s, 1H), 7.27 (s, 2H), 7.40 (d, 2H, J=7.8 Hz), 7.55 (d, 2H, J=7.8 Hz), 7.95 (s, 1H), 8.23 (s, 1H), 9.28 (s, 1H), 11.68 (s, 1H); ¹³C NMR (150 MHz, DMSO-d₆) δ 20.84, 104.44, 105.99, 126.44, 129.50, 130.19, 132.73, 137.49, 138.87, 152.89, 158.44, 165.70; MS (ESI-negative ion) m/z (relative intensity) 333.2 (14.3), 376.1 (100), 753.6 (8)

### Example 11

### 2-(6-bromopyridin-2-yl)-9-(4-methylphenyl)-8-oxo-8,9-dihydro-7H-purine-6-carboxamide

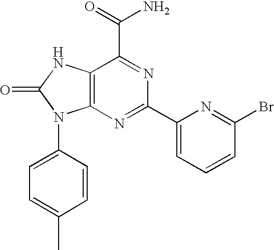

IR: 3429.1, 3308.5, 3173.8, 1744.3, 1685.0, 1625.9, 1596.0, 1575.9, 1518.6, 1474.7, 1414.2, 1364.8, 1227.0, 1180.8, 1122.8, 1002.3, 872.9, 823.5, 686.2, 707.6, 719.4, 731.7, 749.9 cm⁻¹; ¹H NMR (600 MHz, DMSO-d₆) δ 2.42 (s, 3H), 7.40 (d, 2H, J=7.8 Hz), 7.54 (d, 2H, J=7.8 Hz), 7.71 (d, 1H, J=7.8 Hz), 7.88 (t, 1H, J=7.8 Hz), 8.01 (s, 1H), 8.40 (s, 1H), 8.67 (s, 1H), 11.90 (s, 1H); ¹³C NMR (150 MHz, DMSO-d₆) δ 20.90, 121.02, 123.22, 126.89, 128.89, 129.63, 130.05, 132.72, 137.95, 140.23, 141.22, 152.83, 153.21, 153.46, 155.52, 165.46; MS (ESI-negative ion) m/z (relative intensity) 379.9 (18), 381.9 (18), 423.3 (86), 425.3 (100).

### Example 12

### 4-[6-(aminocarbonyl)-9-(4-methylphenyl)-8-oxo-8,9-dihydro-7H-purin-2-yl]-2,6-dimethoxyphenyl acetate

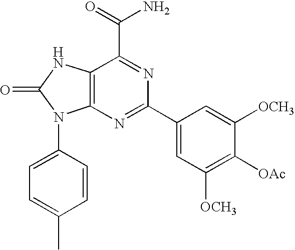

IR: 1725.8, 1669.3, 1611.1, 1518.8, 1473.0, 1389.6, 1219.9, 1128.8, 1055.6, 1033.3, 874.2, 771.2 cm⁻¹; ¹H NMR (600 MHz, DMSO-d₆) δ 2.27 (s, 3H), 2.40 (s, 3H), 3.85 (s, 6H), 7.39 (d, 2H, J=7.8 Hz), 7.67 (d, 2H, J=7.8 Hz), 7.78 (s, 2H), 8.01 (s, 1H), 8.57 (s, 1H), 11.77 (s, 1H); ¹³C NMR (150 MHz, DMSO-d₆) δ 20.26, 20.86, 56.21, 104.49, 120.03, 125.67, 129.27, 130.19, 132.96, 135.25, 137.21, 151.91, 152.66, 152.90, 153.97, 165.66, 168.04; MS (ESI-negative ion) m/z (relative intensity) 462.4 (100).

### Example 13

### 2-(2-chloro-6-nitrophenyl)-9-(4-methylphenyl)-8-oxo-8,9-dihydro-7H-purine-6-carboxamide

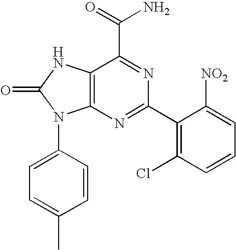

IR: 3444.3, 3205.6, 2924.0, 1741.0, 1677.6, 1597.6, 1572.4, 1516.6, 1463.9, 1424.7, 1387.0, 1247.3, 1161.3, 1032.9, 1002.2, 873.8, 814.0, 770.4, 752.3, 716.9, 683.3 cm⁻¹; ¹H NMR (600 MHz, DMSO-d₆) δ 2.36 (s, 3H), 7.34 (d, 2H, J=8.4 Hz), 7.46 (d, 211, J=8.4 Hz), 7.75 (t, 11-1, J=7.8 Hz), 7.94 (s, 1H), 8.00 (d, 1H, J=8.4 Hz), 8.03 (s, 1H), 8.12 (d, 1H, J=8.4 Hz), 11.97 (s, 1H); ¹³C NMR (150 MHz, DMSO-d₆) δ 20.7, 120.32, 123.38, 126.14, 129.49, 129.57, 131.15, 131.99, 132.78, 134.07, 134.91, 137.81, 149.47, 151.74, 152.58, 152.75, 164.91; MS (ESI-negative ion) m/z (relative intensity) 380.3 (18), 423.3 (100).

### Example 14

### 2-(2-fluoropyridin-3-yl)-9-(4-methylphenyl)-8-oxo-8,9-dihydro-7H-purine-6-carboxamide

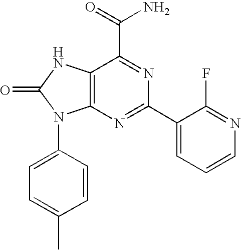

IR: 3461.1, 3329.1, 3037.5, 1732.5, 1682.4, 1590.3, 1569.6, 1518.5, 1444.9, 1371.6, 1266.6, 1218.8, 1168.3, 1026.6, 1002.9, 903.4, 873.7, 812.0, 769.6, 743.8, 716.4, 654.6 cm⁻¹; ¹H NMR (600 MHz, DMSO-d₆) δ 2.29 (s, 3H), 7.38 (d, 2H, J=7.8 Hz), 7.49 (t, 1H, J=6.0 Hz), 7.58 (d, 2H, J=7.8 Hz), 8.02 (s, 1H), 8.29 (s, 1H), 8.33 (d, 1H, J=3.6 Hz), 8.77 (t, 1H, J=3.0 Hz), 11.91 (s, 1H); ¹³C NMR (150 MHz, DMSO-d₆) δ 20.73, 120.07, 120.43, 120.58, 122.11, 122.14, 126.03, 129.34, 129.90, 132.76, 137.45, 142.69, 148.33, 148.43, 151.14, 151.20, 152.72, 152.90, 159.46, 161.08, 165.24; MS (ESI-negative ion) m/z (relative intensity) 363.2 (76), 320.3 (30).

### Example 15

### 2-(3,4-dimethoxyphenyl)-9-(4-methylphenyl)-8-oxo-8,9-dihydro-7H-purine-6-carboxamide

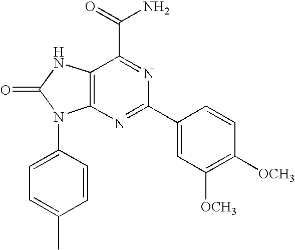

IR: 3450.2, 3100.8, 1732.1, 1681.5, 1599.0, 1572.7, 1518.1, 1461.7, 1392.1, 1343.7, 1269.6, 1226.9, 1164.5, 1122.6, 1025.0, 1000.5, 898.2, 815.1, 771.1, 751.5, 735.6, 695.4 cm⁻¹; ¹H NMR (600 MHz, DMSO-d₆) δ 2.41 (s, 3H), 3.81 (s, 3H), 3.86 (s, 3H), 7.02 (d, 1H, J=9.0 Hz), 7.40 (d, 2H, J=7.2 Hz), 7.60 (d, 2H, J=7.8 Hz), 7.96 (m, 3H), 8.02 (s, 1H), 8.50 (s, 1H), 11.66 (s, 1H); ¹³C NMR (150 MHz, DMSO-d₆) δ 20.76, 55.51, 55.75, 111.09, 111.32, 119.27, 120.79, 125.97, 129.29, 129.62, 130.11, 132.88, 137.24, 148.65, 150.75, 152.71, 152.84, 154.72, 165.68; MS (ESI-negative ion) m/z (relative intensity) 404.3 (100).

### Example 16

### 2-(4-chloro-3-nitrophenyl)-9-(4-methylphenyl)-8-oxo-8,9-dihydro-7H-purine-6-carboxamide

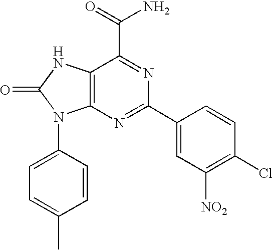

IR: 3450.0, 3213.0, 1744.5, 1684.7, 1596.4, 1567.1, 1518.7, 1470.5, 1416.5, 1381.2, 1342.3, 1170.5, 1052.8, 1003.9, 881.3, 808.6, 718.3 cm⁻¹; ¹H NMR (600 MHz, DMSO-d₆) δ 2.39 (s, 3H), 7.41 (d, 2H, J=7.8 Hz), 7.57 (d, 2H, J=7.8 Hz), 7.86 (d, 1H, J=9.0 Hz), 8.01 (s, 1H), 8.59 (d, 1H, J=8.4 Hz), 8.74 (s, 1H), 9.13 (s, 1H), 11.91 (s, 1H); ¹³C NMR (150 MHz, DMSO-d₆) δ 20.76, 123.73, 125.60, 126.28, 129.47, 129.82, 131.60, 131.66, 132.61, 137.44, 137.63, 148.46, 151.44, 153.07, 165.33; MS (ESI-negative ion) m/z (relative intensity) 380.4 (19), 424.3 (100).

### Example 17

### 2-[3-fluoro-5-(trifluoromethyl)phenyl]-9-(4-methylphenyl)-8-oxo-8,9-dihydro-7H-purine-6-carboxamide

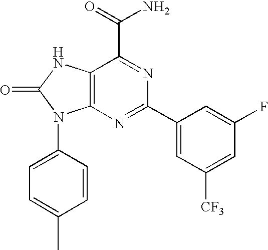

IR: 3502.8, 3386.6, 3197.6, 1747.9, 1684.1, 1606.9, 1568.3, 1517.3, 1467.0, 1392.7, 1316.7, 1308.7, 1169.7, 1132.36, 1002.8, 898.4, 870.6, 812.8, 768.0, 715.2, 692.2, 665.1 cm⁻¹; ¹H NMR (600 MHz, DMSO-d₆) δ 2.39 (s, 3H), 7.41 (d, 2H, J=8.4 Hz), 7.59 (d, 2H, J=7.8 Hz), 7.77 (d, 1H, J=8.4 Hz), 7.99 (s, 1H), 8.54 (s, 1H), 8.62 (d, 1H, J=9.6 Hz), 8.79 (s, 1H), 11.90 (s, 1H); ¹³C NMR (150 MHz, DMSO-d₆) δ 20.76, 113.93, 114.13, 118.21, 118.36, 119.87, 120.85, 126.12, 129.40, 129.81, 131.19, 131.41, 132.74, 137.62, 140.83, 140.89, 151.68, 152.89, 152.91, 161.61, 163.24, 165.36; MS (ESI-negative ion) m/z (relative intensity) 387.2 (52), 430.4 (100).

### Example 18

### 2-(3-formylphenyl)-9-(4-methylphenyl)-8-oxo-8,9-dihydro-7H-purine-6-carboxamide

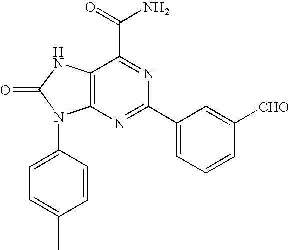

IR: 3152.1, 1741.3, 1681.1, 1596.4, 1576.0, 1518.5, 1468.2, 1414.7, 1366.0, 1263.7, 1228.5, 1151.3, 1121.8, 1003.6, 873.1, 807.1, 732.9, 709.0 cm⁻¹; ¹H NMR (600 MHz, DMSO-d₆) δ 2.43 (s, 3H), 7.34 (d, 2H, J=7.8 Hz), 7.63 (d, 2H, J=7.8 Hz), 7.71 (t, 1H, J=7.8 Hz), 7.98 (d, 1H, J=7.2 Hz), 8.04 (s, 1H), 8.59 (s, 1H), 8.77 (d, 1H, J=7.8 Hz), 8.95 (s, 1H), 10.11 (s, 1H), 11.83 (s, 1H); ¹³C NMR (150 MHz, DMSO-d₆) δ 20.78, 120.3, 125.93, 126.29, 129.04, 129.30, 129.39, 129.47, 129.94, 130.18, 132.74, 133.29, 136.58, 137.56, 137.83, 152.94, 153.05, 153.39, 165.50; MS (ESI-negative ion) m/z (relative intensity) 372.3 (19).

### Example 19

### 9-(4-methylphenyl)-8-oxo-2-phenyl-8,9-dihydro-7H-purine-6-carboxamide

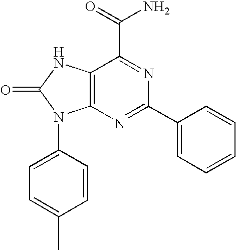

IR: 3450.6, 3163.2, 1723.3, 1683.3, 1666.8, 1598.8, 1574.7, 1518.4, 1465.4, 1415.9, 1390.0, 1227.5, 1182.0, 1165.4, 1130.9, 1052.6, 1005.9, 1025.1, 896.8, 870.2, 815.8, 766.9, 753.7, 896.0 cm⁻¹; ¹H NMR (600 MHz, DMSO-d₆) δ 2.41 (s, 3H), 7.40 (d, 2H, J=8.4 Hz), 7.46 (m, 3H), 7.7.58 (d, 2H, J=7.8 Hz), 7.96 (s, 1H), 8.43 (m, 2H), 8.48 (s, 1H), 11.73 (s, 1H); ¹³C NMR (150 MHz, DMSO-d₆) δ 21.99, 127.43, 128.78, 129.63, 130.64, 131.26, 131.29, 134.06, 138.09, 138.64, 154.16, 155.80, 166.84; MS (ESI-negative ion) m/z (relative intensity) 301.1 (11), 344.3 (73).

### Example 20

### 2-cyclohexyl-9-(4-methylphenyl)-8-oxo-8,9-dihydro-7H-purine-6-carboxamide

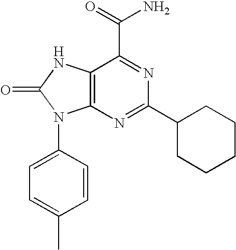

IR: 3446.1, 3163.0, 2923.2, 2853.4, 1728.5, 1683.7, 1598.5, 1574.6, 1515.4, 1454.5, 1415.9, 1391.8, 1166.6, 1130.7, 1000.8, 872.6, 817.2, 697.0 cm⁻¹; ¹H NMR (600 MHz, DMSO-d₆) δ 1.22 (m, 1H), 1.32 (m, 2H), 1.55 (m, 2H), 1.66 (d, 1H, J=12.0 Hz), 1.75 (d, 2H, J=13.2 Hz), 1.91 (d, 2H, J=11.2 Hz), 2.39 (s, 3H), 2.70 (m, 1H), 7.36 (d, 2H, J=8.4 Hz), 7.48 (d, 2H, J=7.8 Hz), 7.87 (s, 1H), 8.05 (s, 1H), 11.53 (s, 1H); ¹³C NMR (150 MHz, DMSO-d₆) δ; MS (ESI-negative ion) m/z (relative intensity) 307.2 (14), 350.3 (93).

Illustrative of Scheme 2 is the synthesis of Examples 21-38.

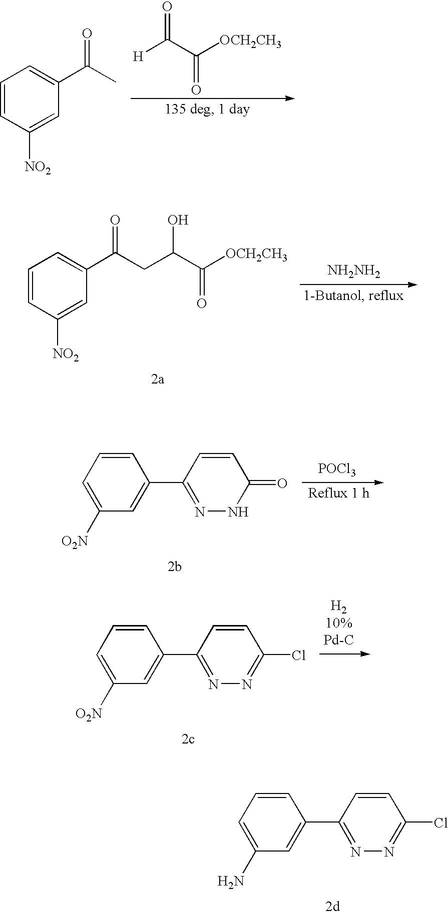

### Step 1

### Preparation of Compound 2a

A mixture of 50% ethyl glyoxylate in toluene ((13 mL g, 60.6 mmol) and o-nitroacetophene (10 g, 60.6 mmol) was heated in an oil bath at 135° C. for 24 h. The crude residue was chromatographed (40:60 EtOAc:Hexanes). Pure ethyl 13-(o-nitrobenzoyl)lactate (Compound 2a) was obtained in 40% yield.

### Step 2

### Preparation of Compound 2b

A solution of ethyl β-(o-nitrobenzoyl)lactate (2 g, 7.5 mmol) Compound 2b in 20 mL of 1-butanol was treated with hydrazine hydrate (0.35 mL, 9.3 mmol), and the reaction mixture was refluxed overnight. After cooling, the pyridazinone (Compound 2b) crystallized. Pure pyridazinone was collected by filtering, and washed with some 1-butanol with yield of 85%.

### Step 3

### Preparation of Compound 2c

Pyridazinone, Compound 2b, (2 g, 9.2 mmol) was added to a flask containing 25 ml POCl₃. The solution was refluxed for 1 h. POCl₃ was removed. Add ice-water slowly to the residue. Collect the precipitation. The crude residue was chromatographed (1:50 MeOH:CH₂Cl₂). 3-Chloro-6-(3-nitrophenyl)pyridazine (Compound 2c) was obtained in 91% yield.

### Step 4

### Preparation of Compound 2d

3-Chloro-6-(3-nitrophenyl)pyridazine, Compound 2c, (1.8 g, 7.6 mmol) dissolved in 180 mL EtOH is hydrogenated at room temperature in the presence of 300 mg of 10% palladium on charcoal. After 1 h, the catalyst is filtered off, the filtrate is concentrated. The residue was chromatographed (1:30 MeOH:CH₂Cl₂) to obtain 3-Chloro-6-(3-aminophenyl)pyridazine (Compound 2d) in 55% yield.

### General Procedures

### Preparation of N-[3-(6-chloropyridazin-3-yl)phenyl]-1,3-benzodioxole-5-carboxamide

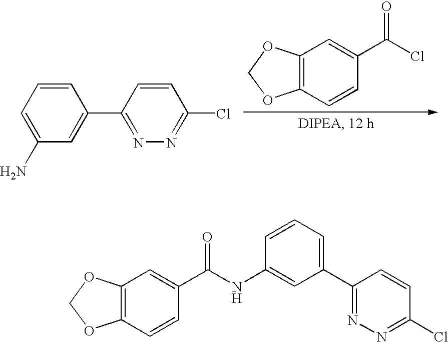

2d

General Procedure A. To a solution of 3-Chloro-6-(3-aminophenyl)pyridazine Compound 2d and piperonyloyl chloride in 10 mL CH₂Cl₂ was added DIPEA at 0° C. The mixture was allowed to warm to room temperature, and stirred overnight. Add 50 mL to the mixture, and wash the organic phase with water, brine. The organic phase was dried over Na₂SO₄, filtered and evaporated. Flash chromatography of the crude product (1:30 MeOH:CH₂Cl₂) afforded N-[3-(6-chloropyridazin-3-yl)phenyl]-1,3-benzodioxole-5-carboxamide in 80%. ¹H NMR (DMSO-d6) δ 9.89 (s, 1H), 8.46 (s, 1H), 7.97 (d, J=7.8 Hz, 1H), 7.9 (d, J=9.0 Hz, 1H), 7.69 (d, J=7.8 Hz, 1H), 7.60 (d, J=9.0 Hz, 1H), 7.56 (d, J=8.4 Hz, 1H), 7.50 (s, 1H), 7.41 (t, J=8.4 Hz, 1H), 6.81 (d, J=7.8 Hz, 1H), 5.99 (s, 2H). ESI-MS m/z 354 (MH⁺).

### Preparation of N-[3-(6-chloropyridazin-3-yl)phenyl]-3-nitrobenzamide

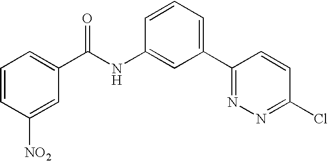

Prepared according to General Procedure A. ¹H NMR (DMSO-d6) δ 10.81 (s, 1H), 8.86 (s, 1H), 8.64 (s, 1H), 8.45-8.47 (m, 2H), 8.32 (d, J=9.0 Hz, 1H), 8.05 (d, J=9.0 Hz, 2H), 7.86-7.89 (m, 2H), 7.60 (t, J=7.8 Hz, 1H). ESI-MS m/z 355 (MH⁺).

### Preparation of N-[3-(6-chloropyridazin-3-yl)phenyl]-3,4,5-trimethoxybenzamide

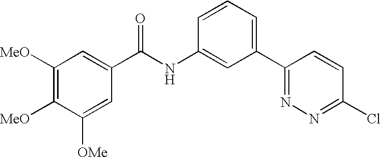

Prepared according to General Procedure A. ¹H NMR (DMSO-d6) δ 10.34 (s, 1H), 8.57 (s, 1H), 8.31 (d, J=9.0 Hz, 1H), 8.02-8.05 (m, 2H), 7.85 (d, J=7.8 Hz, 1H), 7.57 (t, J=7.8 Hz, 1H), 7.34 (s, 2H), 3.89 (s, 6H), 3.74 (s, 3H). ESI-MS m/z 400 (MH⁺).

### Preparation of N-[3-(6-chloropyridazin-3-yl)phenyl]-3,4-dimethoxybenzamide

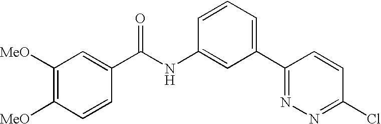

Prepared according to General Procedure A. ¹H NMR (DMSO-d6) δ 10.29 (s, 1H), 8.60 (s, 1H), 8.30 (d, J=9.0 Hz, 1H), 8.03 (d, J=9.0 Hz, 2H), 7.82 (d, J=7.8 Hz, 1H), 7.68 (d, J=8.4 Hz, 1H), 7.59 (s, 1H), 7.55 (t, J=7.8 Hz, 1H), 7.11 (d, J=7.8 Hz, 1H), 3.86 (s, 3H), 3.85 (s, 3H). ESI-MS m/z 370 (MH⁺).

### Preparation of 3-nitro-N-[3-(3-phenyl[1,2,4]triazolo[4,3-b]pyridazin-6-yl)phenyl]benzamide

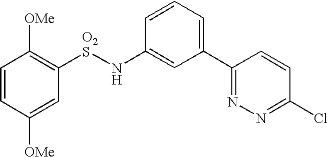

Prepared according to General Procedure A. ¹H NMR (DMSO-d6) δ 10.01 (s, 1H), 7.88-7.92 (m, 2H), 7.70-7.71 (m, 1H), 7.58 (d, J=6.6 Hz, 1H), 7.25-7.31 (m, 3H), 6.96-6.98 (m, 1H), 6.90-6.92 (m, 1H), 3.8 (s, 3H), 3.69 (s, 3H). ESI-MS m/z 406 (MH⁺).

The invention is further illustrated by the following examples.

### Example 21

### N-[3-(3-thien-2-yl[1,2,4]triazolo[4,3-b]pyridazin-6-yl)phenyl]-1,3-benzodioxole-5-carboxamide

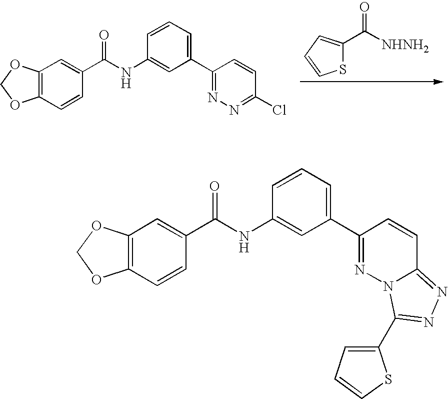

Prepared according to General Procedure B. A suspension of 2-Thiophenecarbohydrazide (98 mg, 0.69 mmol) and N-[3-(6-chloropyridazin-3-yl)phenyl]-1,3-benzodioxole-5-carboxamide (90 mg, 0.28 mmol) in 4 mL 1-butanol was heated at 140° C. inside a microwave for 4 h. Remove the solvent. Flash chromatography of the crude product (1:30 MeOH:CH₂Cl₂) afforded N-[3-(3-thien-2-yl[1,2,4]triazolo[4,3-b]pyridazin-6-yl)phenyl]-1,3-benzodioxole-5-carboxamide in 75% yield. ¹H NMR (DMSO-d6) δ 10.34 (s, 1H), 8.66 (s, 1H), 8.56 (d, J=9.6 Hz, 1H), 8.35 (d, J=3.6 Hz, 1H), 8.00 (d, J=7.8 Hz, 1H), 7.89-7.96 (m, 3H), 7.60-7.75 (m, 2H), 7.57 (s, 1H), 7.39 (t, J=4.5 Hz, 1H), 7.10 (d, J=7.8 Hz, 1H), 6.16 (s, 2H). ESI-MS m/z 442 (MH⁺).

### Example 22

### 3,4,5-trimethoxy-N-[3-(3-phenyl[1,2,4]triazolo[4,3-b]pyridazin-6-yl)phenyl]benzamide

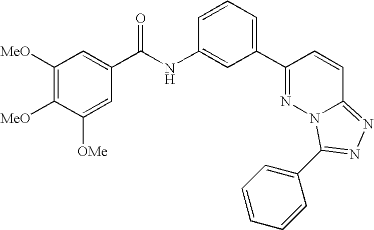

Prepared according to General Procedure B. ¹H NMR (CDCl₃) δ 9.01 (s, 1H), 8.44 (d, J=7.2 Hz, 2H), 8.36 (s, 1H), 7.95 (d, J=9.6 Hz, 1H), 7.86 (d, J=7.8 Hz, 1H), 7.70 (d, J=7.8 Hz, 1H), 7.52 (d, J=9.6 Hz, 1H), 7.45-7.48 (m, 3H), 7.42 (d, J=7.2 Hz, 1H), 7.20 (s, 2H), 3.87 (s, 3H), 3.85 (s, 6H). ESI-MS m/z 482 (MH⁺).

### Example 23

### 3,4,5-trimethoxy-N-(3-[1,2,4]triazolo[4,3-b]pyridazin-6-ylphenyl)benzamide

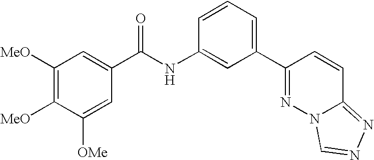

Prepared according to General Procedure B. ¹H NMR (DMSO-d6) δ 10.38 (s, 1H), 9.73 (s, 1H), 8.49-8.52 (m, 2H), 8.00 (d, J=7.8 Hz, 1H), 7.93 (d, J=9.6 Hz, 1H), 7.84 (d, J=7.8 Hz, 1H), 7.60 (t, J=7.8 Hz, 1H), 7.5 (s, 2H), 3.89 (s, 6H), 335 (s, 3H). ESI-MS m/z 406 (MH⁺).

### Example 24

### 3,4,5-trimethoxy-N-[3-(3-thien-2-yl[1,2,4]triazolo[4,3-b]pyridazin-6-yl)phenyl]benzamide

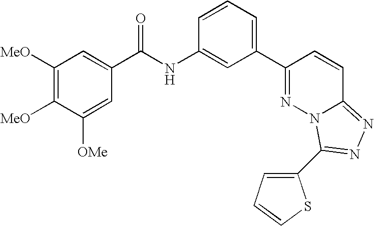

Prepared according to General Procedure B. NMR (DMSO-d6) δ 10.42 (s, 1H), 8.63 (s, 1H), 8.56 (d, J=9.6 Hz, 1H), 8.35 (m, 1H), 7.95-8.00 (m, 3H), 7.87-7.90 (m, 1H), 7.64 (t, J=8.1 Hz, 1H), 7.37-7.7.38 (m, 3H), 3.90 (s, 6H), 3.75 (s, 3H). ESI-MS m/z 488 (MH⁺).

### Example 25

### 3,4,5-trimethoxy-N-[3-(3-pyridin-3-yl[1,2,4]triazolo[4,3-b]pyridazin-6-yl)phenyl]benzamide

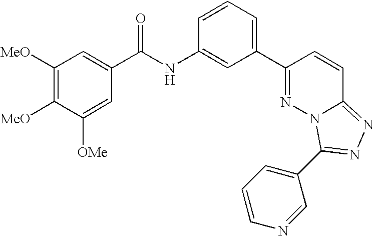

Prepared according to General Procedure B. ¹H NMR (DMSO-d6) δ 10.39 (s, 1H), 9.60 (s, 1H), 8.84 (d, J=7.8 Hz, 1H), 8.76 (d, J=3.6 Hz, 1H), 8.58 (d, J=10.2 Hz, 1H), 8.54 (s, 1H), 7.96-7.97 (m, 2H), 7.87 (d, J=8.4 Hz, 1H), 7.67-7.69 (m, 1H), 7.60 (t, J=7.8 Hz, 1H), 7.31 (s, 2H), 3.89 (s, 6H), 3.75 (s, 3H). ESI-MS m/z 483 (MH⁺).

### Example 26

### 3,4,5-trimethoxy-N-{3-[3-(1H-pyrrol-2-yl)[1,2,4]triazolo[4,3-b]pyridazin-6-yl]phenyl}benzamide

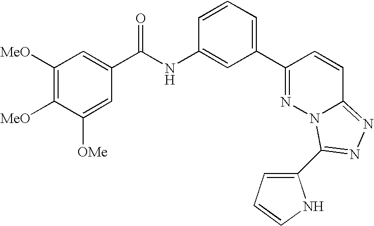

Prepared according to General Procedure B. ¹H NMR (DMSO-d6) δ 12.03 (s, 1H), 10.42 (s, 1H), 8.59 (s, 1H), 8.54 (d, J=60.6 Hz, 1H), 7.99-8.00 (m, 1H), 7.89-7.94 (m, 2H), 7.63 (t, J=8.0 Hz, 1H), 7.39 (s, 1H), 7.33 (s, 2H), 7.08 (s, 1H), 6.34 (d, J=2.4 Hz, 1H), 3.90 (s, 6H), 3.75 (s, 3H). ESI-MS m/z 471 (MH⁺).

### Example 27

### 3-nitro-N-[3-(3-phenyl[1,2,4]triazolo[4,3-b]pyridazin-6-yl)phenyl]benzamide

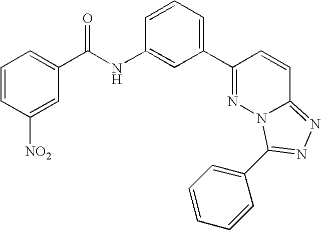

Prepared according to General Procedure B. ¹H NMR (DMSO-d6) δ 10.84 (s, 1H), 8.85 (s, 1H); 8.69 (s, 1H); 8.53-8.57 (m, 3H), 8.45-8.47 (m, 2H), 7.87-8.00 (m, 4H), 7.63-7.66 (m, 4H). ESI-MS m/z 437 (MH⁺).

### Example 28

### 3-nitro-N-(3-[1,2,4]triazolo[4,3-b]pyridazin-6-ylphenyl)benzamide

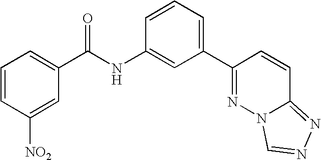

Prepared according to General Procedure B. ¹H NMR (DMSO-d6) δ 10.84 (s, 1H), 9.73 (s, 1H), 8.86 (s, 1H), 8.57 (s, 1H), 8.45-8.51 (m, 3H), 8.03 (d, J=7.8 Hz, 1H), 7.93 (d, J=9.6 Hz, 1H), 7.86-7.89 (m, 2H), 7.63 (t, J=7.8 Hz, 1H). ESI-MS m/z 361 (MH⁺).

### Example 29

### 3-nitro-N-[3-(3-thien-2-yl[1,2,4]triazolo[4,3-b]pyridazin-6-yl)phenyl]benzamide

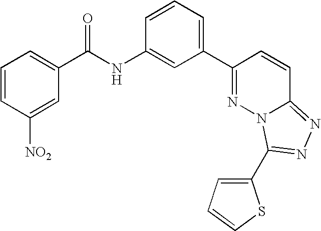

Prepared according to General Procedure B. ¹H NMR (DMSO-d6) δ 10.86 (s, 1H), 8.85 (s, 1H); 8.69 (s, 1H); 8.57 (d, J=9.6 Hz, 1H), 8.46-8.48 (m, 2H), 8.35 (d, J=2.4 Hz, 1H), 7.97-7.99 (m, 2H), 7.87-7.90 (m, 3H), 7.66 (t, J=7.8 Hz, 1H), 7.22 (s, 1H). ESI-MS m/z 443 (MH⁺).

### Example 30

### 3-nitro-N-{3-[3-(1H-pyrrol-2-yl)[1,2,4]triazolo[4,3-b]pyridazin-6-yl]phenyl}benzamide

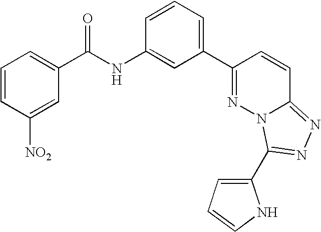

Prepared according to General Procedure B. ¹H NMR (DMSO-d6) δ 12.04 (s, 1H), 10.87 (s, 1H), 8.85 (s, 1H); 8.65 (s, 1H); 8.45-8.49 (m, 3H), 8.04 (d, J=7.8 Hz, 2H), 7.96 (d, J=7.2 Hz, 1H), 7.87-7.91 (m, 2H), 7.65 (t, J=7.8 Hz, 1H), 7.40 (s, 1H), 7.08 (s, 1H), 6.35 (s, 1H). ESI-MS m/z 426 (MH⁺).

### Example 31

### 3,4-dimethoxy-N-[3-(3-phenyl[1,2,4]triazolo[4,3-b]pyridazin-6-yl)phenyl]benzamide

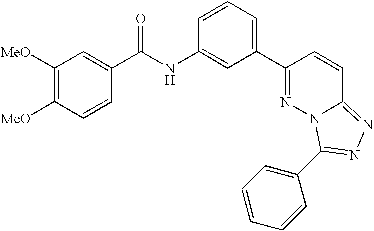

Prepared according to General Procedure B. ¹H NMR (DMSO-d6) δ 10.36 (s, 1H), 8.65 (s, 1H), 8.54-8.58 (m, 3H), 8.01 (d, J=8.4 Hz, 1H), 7.87 (d, J=10.2 Hz, 1H), 7.89 (d, J=7.8 Hz, 1H), 7.67-7.70 (m, 3H), 7.60-7.63 (m, 3H), 7.14 (d, J=8.4 Hz, 1H), 3.87 (s, 3H), 3.86 (s, 3H). ESI-MS m/z 452 (MH⁺).

### Example 32

### 3,4-dimethoxy-N-[3-(3-thien-2-yl[1,2,4]triazolo[4,3-b]pyridazin-6-yl)phenyl]benzamide

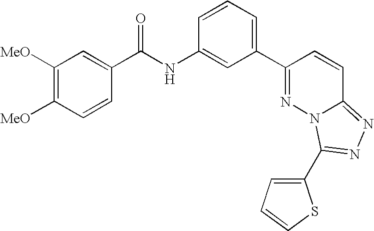

Prepared according to General Procedure B. ¹H NMR (DMSO-d6) δ 10.36 (s, 1H), 8.64 (s, 1H), 8.55-8.57 (m, 1H), 8.35 (d, J=3.0 Hz, 1H), 8.01 (d, J=7.8 Hz, 1H), 7.93-7.97 (m, 2H), 7.90 (d, J=4.8 Hz, 1H), 7.67-7.69 (m, 1H), 7.61-7.64 (m, 2H), 7.37-7.38 (m, H), 7.12-7.13 (m, 1H), 3.87 (s, 3H), 3.86 (s, 3H). ESI-MS m/z 458 (MH⁺).

### Example 33

### 3,4-dimethoxy-N-[3-(3-pyridin-3-yl[1,2,4]triazolo[4,3-b]pyridazin-6-yl)phenyl]benzamide

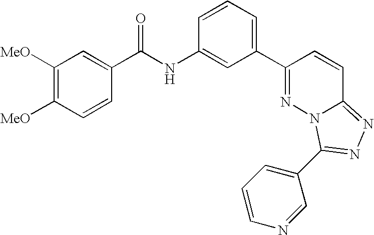

Prepared according to General Procedure B. ¹H NMR (DMSO-d6) δ 10.35 (s, 1H), 9.63 (s, 1H), 8.86-8.87 (d, J=7.8 Hz, 1H), 8.78 (s, 1H), 8.59 (t, J=9.6 Hz, 2H), 7.98-8.01 (m, 2H), 7.88 (d, J=7.8 Hz, 1H), 7.67-7.72 (m, 2H), 7.58-7.62 (m, 2H), 7.12 (d, J=8.4 Hz, 1H), 3.87 (s, 3H), 3.86 (s, 3H). ESI-MS m/z 453 (MH⁺).

### Example 34

### 3,4-dimethoxy-N-[3-(3-pyridin-4-yl[1,2,4]triazolo[4,3-b]pyridazin-6-yl)phenyl]benzamide

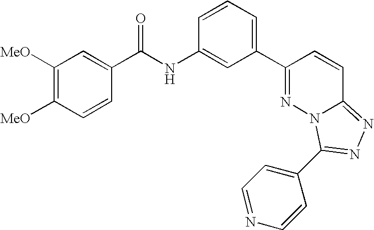

Prepared according to General Procedure B. ¹H NMR (DMSO-d6) δ 10.35 (s, 1H), 8.88 (m, 2H), 8.69 (s, 1H), 8.63 (d, J=9.0 Hz, 1H), 8.52 (m, 2H), 8.01-8.05 (m, 2H), 7.92 (d, J=6.0 Hz, 1H), 7.69-7.70 (d, J=6.6 Hz, 1H), 7.60-7.62 (m, 2H), 7.13-7.14 (m, 1H), 3.88 (s, 3H), 3.86 (s, 3H). ESI-MS m/z 453 (MH⁺).

### Example 35

### 3,4-dimethoxy-N-{3-[3-(1H-pyrrol-2-yl)[1,2,4]triazolo[4,3-b]pyridazin-6-yl]phenyl}benzamide

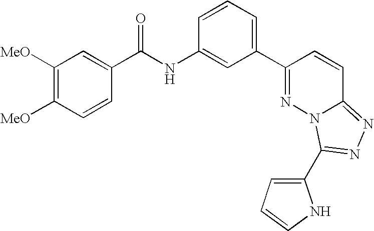

Prepared according to General Procedure B. ¹H NMR (DMSO-d6) δ 12.03 (s, 1H), 10.37 (s, 1H), 8.61 (s, 1H), 8.48 (d, J=10.2 Hz, 1H), 8.02 (d, J=8.4 Hz, 1H), 7.88-7.91 (m, 2H), 7.68 (d, J=8.4 Hz, 1H), 7.59-7.63 (m, 2H), 7.40 (s, 1H), 7.13 (d, J=8.4 Hz, 1H), 7.08 (s, 1H), 6.35 (d, J=4.8 Hz, 1H), 3.87 (s, 3H), 3.86 (s, 3H). ESI-MS m/z 441 (MH⁺).

### Example 36

### 3,4-dimethoxy-N-[3-(3-methyl[1,2,4]triazolo[4,3-b]pyridazin-6-yl)phenyl]benzamide

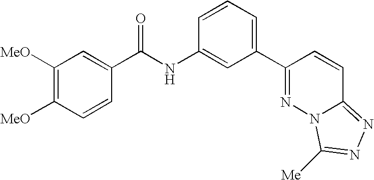

Prepared according to General Procedure B. ¹H NMR (DMSO-d6) δ 10.32 (s, 1H), 8.51 (s, 1H), 8.42 (d, J=9.6 Hz, 1H), 8.00 (d, J=7.8 Hz, 1H), 7.83-7.86 (m, 2H), 7.68 (d, J=8.4 Hz, 1H), 7.56-7.59 (m, 2H), 7.11 (d, J=8.4 Hz, 1H), 3.86 (s, 3H), 3.85 (s, 3H). ESI-MS m/z 390 (MH⁺).

### Example 37

### 2,5-dimethoxy-N-(3-[1,2,4]triazolo[4,3-b]pyridazin-6-ylphenyl)benzenesulfonamide

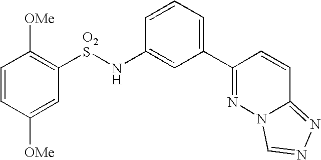

Prepared according to General Procedure B. ¹H NMR (DMSO-d6) δ 10.31 (s, 1H), 9.69 (s, 1H), 8.44 (d, J=9.6 Hz, 1H), 7.84 (s, 1H), 7.77 (d, J=9.6 Hz, 1H), 7.69 (d, J=7.8 Hz, 1H), 7.42 (t, J=7.8 Hz, 1H), 7.29-7.32 (m, 2H), 7.12-7.15 (m, 2H), 3.90 (s, 3H), 3.75 (s, 3H). ESI-MS m/z 412 (MH⁺).

### Example 38

### 2,5-dimethoxy-N-[3-(3-thien-2-yl[1,2,4]triazolo[4,3-b]pyridazin-6-yl)phenyl]benzenesulfonamide

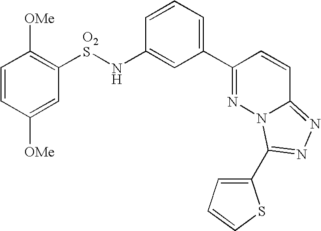

Prepared according to General Procedure B. ¹H NMR (DMSO-d6) δ 10.43 (s, 1H), 8.51 (d, J=9.6 Hz, 1H), 8.35 (m, 1H), 8.09 (s, 1H), 7.89-7.93 (m, 2H), 7.81 (d, J=7.8 Hz, 1H), 7.46 (t, J=7.8 Hz, 1H), 7.40-7.41 (m, 1H), 7.34-7.36 (m, 2H), 7.13 (m, 2H), 3.83 (s, 3H), 3.61 (s, 3H). ESI-MS m/z 494 (MH⁺).

Illustrative of Scheme 3 is the synthesis of Example 39.

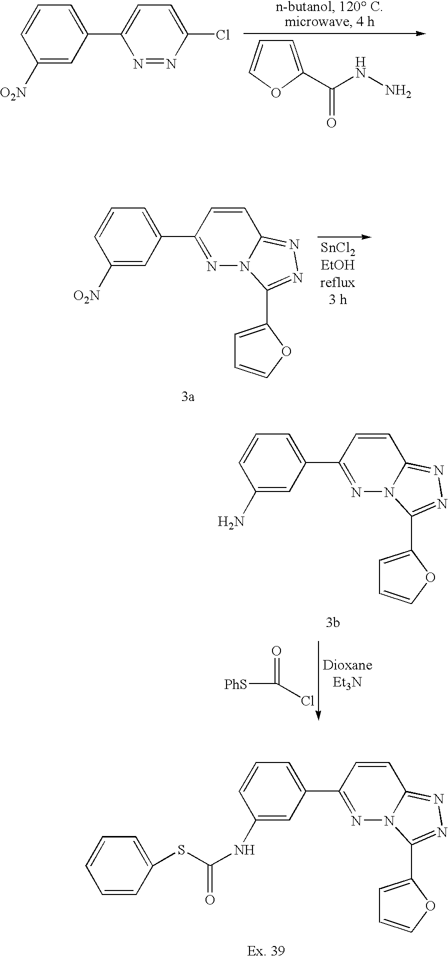

### Step 1

### Preparation of Compound 3a

### 3-(2-furyl)-6-(3-nitrophenyl)[1,2,4]triazolo[4,3-b]pyridazine

A mixture of 3-chloro-6-(3′-nitrophenyl)-pyridazine (242 mg, 1.03 mmol) and 2-furoic hydrazide (260 mg, 2.06 mmol) in n-butanol (4 ml) was heated under microwave irradiation at 120° C. for 4 h. The solvent was evaporated, and the residue was subjected to flash column chromatography on silica gel (elution with CH₂Cl₂-MeOH) to yield 3-(2-furyl)-6-(3-nitrophenyl)[1,2,4]triazolo[4,3-b]pyridazine as a pale yellow solid (126 mg, 40%). ESI-MS: m/z 308 (M+1); ¹H NMR (600 MHz, CDCl₃): δ 8.90 (1H, s), 8.46 (1H, d, J=8.4 Hz), 8.41 (1H, d, J=7.2 Hz), 8.35 (1H, d, J=10.2 Hz), 7.82 (1H, t, J=7.8 Hz), 7.76 (1H, s), 7.68 (1H, d, J=9.6 Hz), 7.59 (1H, d, J=3.6 Hz), 6.72 (1H, m); ¹³C NMR (150.9 MHz, CDCl₃): δ 152.07, 148.97, 144.76, 143.38, 142.51, 140.70, 136.05, 132.98, 130.61, 126.20, 125.68, 122.39, 118.44, 112.83, 111.87.

### Step 2

### Preparation of Compound 3b

### 3-[3-(2-furyl) [1,2,4]triazolo[4,3-b]pyridazin-6-yl]aniline

A solution of 3-(2-furyl)-6-(3-nitrophenyl)[1,2,4]triazolo[4,3-b]pyridazine (125 mg, 0.407 mmol) and anhydrous SnCl₂ (386 mg, 2.03 mmol, 5.0 equiv) in dry ethanol (10 ml) was heated under reflux for 3 h. Upon cooling to rt, ice was added, followed by addition of dichloromethane. The pH of the mixture was adjusted to 7.0 by using a saturated NaHCO₃ aqueous solution. The mixture was filtered, and the solids were washed with warm ethyl acetate. The organic phase was taken out and evaporated to dryness. The residue was subjected to flash column chromatography on silica gel (elution with CH₂Cl₂-MeOH) to yield 3-[3-(2-furyl)[1,2,4]triazolo[4,3-b]pyridazin-6-yl]aniline as a yellow solid (93 mg, 81%). ESI-MS: m/z 278 (M+1); ¹H NMR (600 MHz, CDCl₃-MeOH-d4 85:15 v/v): δ 8.22 (1H, d, J=9.6 Hz), 7.45 (1H, s), 7.66 (1H, d, J=9.6 Hz), 7.65 (1H, d, J=1.8 Hz), 7.35 (3H, m), 6.91 (1H, br. d), 6.69 (1H, m); ¹³C NMR (150.9 MHz, CDCl₃-MeOH-d4 85:15 v/v): δ 154.83, 147.40, 144.57, 143.67, 142.20, 140.61, 135.08, 130.21, 124.63, 120.26, 117.92, 117.62, 113.42, 112.93, 111.71.

### Step 3

### Example 39

### Preparation of S-phenyl 3-[3-(2-furyl)[1,2,4]triazolo[4,3-b]pyridazin-6-yl]phenylthiocarbamate

To a solution of 3-[3-(2-furyl)[1,2,4]triazolo[4,3-b]pyridazin-6-yl]aniline (30 mg, 0.108 mmol) in anhydrous 1,4-dioxane (5 ml) at 0° C. was added phenyl chlorothioformate (43 mg, 0.25 mmol, 2.3 equiv), followed by addition of N,N-diisopropylethylamine (60 μl, 0.34 mmol). The resultant mixture was stirred at rt for 20 h. The formed white solids were collected by filtration to furnish S-phenyl 3-[3-(2-furyl)[1,2,4]triazolo[4,3-b]pyridazin-6-yl]phenylthiocarbamate (21 mg). ESI-MS: m/z 414 (M+1); ¹H NMR (600 MHz, DMSO-d6): δ 10.86 (1H, s, NH), 8.54 (1H, d, J=9.6 Hz), 8.40 (1H, s), 8.06 (1H, s), 7.97 (1H, d, J=9.6 Hz), 7.89 (1H, d, J=7.8 Hz), 7.77 (1H, d, J=8.4 Hz), 7.66 (1H, d, J=3.6 Hz), 7.58 (3H, m), 7.49 (3H, m), 6.82 (1H, m); ¹³C NMR (150.9 MHz, DMSO-d6): δ 163.82, 153.82, 145.51, 144.11, 141.68, 141.00, 140.17, 135.81, 135.07, 130.51, 129.82, 129.59, 128.26, 125.97, 123.26, 121.95, 120.38, 117.97, 112.76, 112.56.

### Preparation of S-phenyl 3-(6-chloropyridazin-3-yl)phenylthiocarbamate

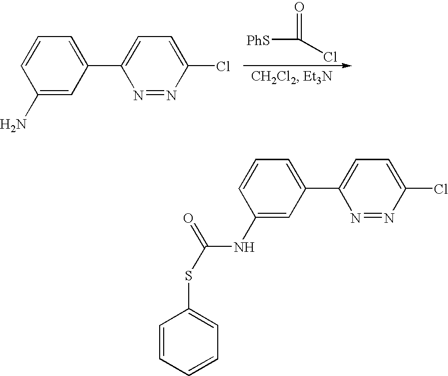

To a suspension of 3-(6′-chloro-3′-pyridazinyl)-benzenamine (1.0 g, 4.86 mmol) in anhydrous dichloromethane (90 ml) at 0° C. was slowly added phenyl chlorothioformate (0.94 g, 5.44 mmol, 1.1 equiv), followed by slow addition of triethylamine (0.81 ml, 5.83 mmol). The resultant solution was stirred at rt for 20 h. After evaporation of volatiles, the residue was subjected to flash column chromatography on silica gel (elution with hexane-EtOAc) to yield S-phenyl 3-(6-chloropyridazin-3-yl)phenylthiocarbamate as a pale yellow solid (820 mg). ESI-MS: m/z 342 (M+1); ¹H NMR (600 MHz, CDCl₃): δ 8.10 (1H, s), 7.82 (1H, d, J=9.0 Hz), 7.80 (1H, d, J=7.8 Hz), 7.63 (2H, m), 7.57 (1H, d, J=7.8 Hz), 7.55 (1H, d, J=9.0 Hz), 7.50-7.43 (4H, m).

### Preparation of phenyl 3-(6-chloropyridazin-3-yl)phenylcarbamate

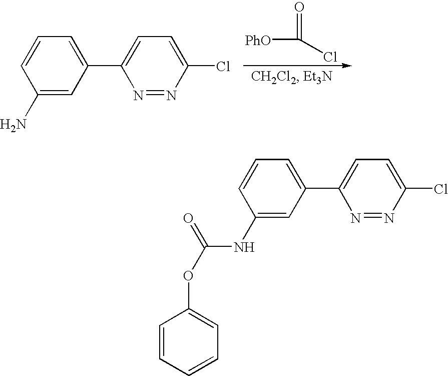

To a solution of 3-(6′-chloro-3′-pyridazinyl)-benzenamine (54 mg, 0.26 mmol) in anhydrous dichloromethane (5 ml) at 0° C. was slowly added phenyl chloroformate (0.94 g, 5.44 mmol, 1.1 equiv), followed by slow addition of triethylamine (45 mg, 0.29 mmol). The resultant solution was stirred at rt for 21 h. After evaporation of volatiles, the residue was subjected to flash column chromatography on silica gel (elution with hexane-EtOAc) to yield phenyl 3-(6-chloropyridazin-3-yl)phenylcarbamate as a pale yellow solid (28 mg, 32%). ESI-MS: m/z 326 (M+1); ¹H NMR (600 MHz, CDCl₃-MeOH-d4 85:15 v/v): δ 8.12 (1H, s), 7.96 (1H, d, J=9.0 Hz), 7.77 (1H, d, J=7.8 Hz), 7.72 (1H, d, J=7.8 Hz), 7.66 (1H, d, J=9.0 Hz), 7.50 (1H, t, J=7.8 Hz), 7.41 (2H, t, J=7.8 Hz), 7.25 (1H, t, J=7.8 Hz), 7.21 (2H, d, J=7.8 Hz); ¹³C NMR (150.9 MHz, CDCl₃-MeOH-d4 85:15 v/v): δ 157.75, 154.84, 151.81, 149.80, 138.45, 134.58, 129.11, 128.52, 128.32, 126.34, 124.82, 121.14, 120.83, 120.00, 116.33.

### In Vitro Kinase Assay

The in vitro kinase assay was performed with baculovirus expressed recombinant cytoplasmic region of the human MET protein (aa 956-1390, NP_{—}000236) using the Z′-LYTE Kinase Assay kit containing Tyr 2 Peptide Substrate (Invitrogen). The assay conditions were as follows: 40 nM recombinant MET, 5 μM ATP, 2 μM Tyr 2 Peptide, 50 mM HEPES (pH 7.5), 10 mM MgCl₂, 1 mM EGTA, 0.01% Brij-35 and the reaction were performed at 30° C. for 90 minutes. Nonphosphorylated peptides were then cleaved by adding the Z′-LYTE Kinase Assay's proprietary development reagent and incubating at room temperature for 60 minutes. Fluorescence was measured using a Packard Fluorocount plate reader using an excitation wavelength of 400 nm and emission wavelengths of 445 nm and 530 nm. A ratio of the 445 nm to 530 nm emission readings are used to quantitate the percent phosphorylation of the peptide.

We claim the following:

1. A compound of the structural Formula I 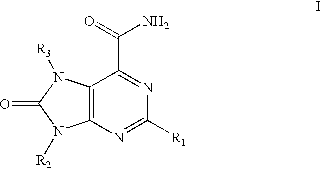
or salt, ester or prodrug thereof, wherein:
R₁ is independently selected from the group consisting of aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₁ is independently alkyl or cycloalkyl;
R₂ is aryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro; and
R₃ is independently selected from the group H, alkenyl, alkoxyalkyl, alkyl, alkynyl, cycloalkyl, cycloalkylalkyl, or haloalkyl;
provided that:
when R₂ is p-tolyl and R₃ is H, R₁ is not phenyl, 3,4-dimethoxyphenyl, 2-thienyl or cyclohexyl.

2. A compound of the structural Formula I 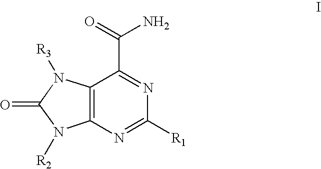
or a salt, ester or prodrug, thereof, wherein:
R₁ is aryl or heteroaryl, optionally substituted by 1-3 substituents independently selected from acyl, acyloxy, alkoxy, halo, haloalkyl, heteroaryl, hydroxy, or nitro, cycloalkyl;
R₂ is aryl, optionally substituted by an alkyl group; and
R₃ is H;
provided that:
when R₂ is p-tolyl and R₃ is H, R₁ is not phenyl, 3,4-dimethoxyphenyl, 2-thienyl or cyclohexyl.

3. A compound selected from the group consisting of Example 2 to Example 14, and Example 16 to Example 18.

4. A compound or composition as recited in claim 1 for use as a medicament.

5. A compound or composition as recited in claim 1 for use in the manufacture of a medicament for the prevent or treatment of a disease of condition ameliorated by the inhibition of c-Met.

6. A pharmaceutical composition as recited in claim 1 useful for the treatment or prevention of a c-Met mediated disease.

7. A pharmaceutical composition comprising a compound recited in claim 3 together with a pharmaceutically acceptable carrier.

8. A method of inhibition of c-Met comprising contacting c-Met with a compound recited in claim 1.

9. A method of treatment of a c-Met-mediated disease comprising the administration of a therapeutically effective amount of a compound recited in claim 1 to a patient in need thereof.

10. The method as recited in claim 9 wherein the disease is cancer.

11. A method for achieving an effect in a patient of c-Met inhibition comprising the administration of a therapeutically effective amount of a compound recited in claim 1 to a patient, wherein the effect is selected from the group consisting of Examples 1 through 20.

12. A compound of the structural Formula II 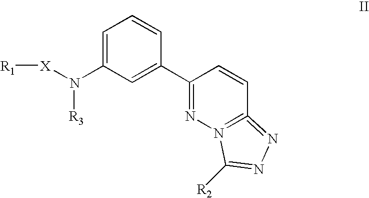
or salt, ester or prodrug, thereof, wherein:
R₁ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, methylenedioxy, or nitro;
R₂ is independently aryl or heteroaryl, optionally substituted by 1-3 substituents independently selected from acyl, acylamino, acyloxy, alkenyl, alkoxy, alkoxyalkyl, alkoxycarbonyl, alkoxycarbonylalkyl, alkyl, alkylaminocarbonyl, alkylcarbonylalkyl, alkylthio, alkylthioalkyl, alkylsulfinyl, alkylsulfinylalkyl, alkynyl, amino, aminocarbonyl, aminoalkyl, aminocarbonylalkyl, aryl, arylamino, arylsulfonyl, arylthio, aralkyl, carboxy, carboxyalkyl, cyano, cycloalkyl, halo, haloalkyl, heteroaryl, heteroaralkyl, heterocyclo, heterocyclocarbonyl, hydroxy, hydroxyalkyl, or nitro;
R₃ is independently selected from the group H, alkenyl, alkoxyalkyl, alkyl, alkynyl, cycloalkyl, cycloalkylalkyl, or haloalkyl; and
X is independently selected from the group CO, SO2, SCO, or OCO;
provided that:
when R₁ is 5-(1,3-benzodioxole), 3,4,5-trimethoxyphenyl, 3-nitrophenyl or 3,4-dimethoxyphenyl and R₃ is H, and X is CO then R₂ is not H, 2-thienyl, 3-pyridyl, 4-pyridyl or methyl;
further provided that:
when R₁ is 2,5-dimethoxyphenyl, R₃ is H and X is SO2, R₂ is not H or 2-thienyl.

13. A compound of the structural Formula II 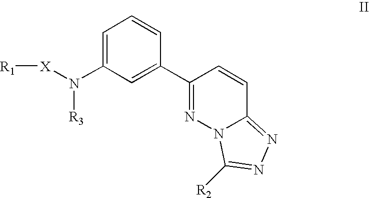
or salt, ester or prodrug, thereof, wherein:
R₁ is aryl, optionally substituted by 1-3 substituents independently selected from alkoxy, methylenedioxy, or nitro;
R₂ is independently aryl or heteroaryl;
R₃ is H; and
X is independently selected from the group CO, SO2, SCO, or OCO;
provided that:
when R₁ is 5-(1,3-benzodioxole), R₃ is H and X is CO, R₂ is not 2-thienyl; when R₁ is 3,4,5-trimethoxyphenyl, R₃ is H and X is CO, R₂ is not H, 2-thienyl or 3-pyridyl; R₁ is 3-nitrophenyl, R₃ is H and X is CO, R₂ is not H or 2-thienyl; R₁ is 3,4-dimethoxyphenyl, R₃ is H and X is CO, R₂ is not 2-thienyl, 3-pyridyl, 4-pyridyl or methyl; R₁ is 2,5-dimethoxyphenyl, R₃ is H and X is SO2, R₂ is not H or 2-thienyl.

14. A compound selected from the group consisting of Examples 22, 26, 27, 30, 31, 35 and 39.

15. A compound or composition as recited in claim 12 for use as a medicament.

16. A compound or composition as recited in claim 12 for use in the manufacture of a medicament for the prevent or treatment of a disease of condition ameliorated by the inhibition of c-Met.

17. A pharmaceutical composition as recited in claim 12 useful for the treatment or prevention of a c-Met mediated disease.

18. A pharmaceutical composition comprising a compound recited in claim 14 together with a pharmaceutically acceptable carrier.

19. A method of inhibition of c-Met comprising contacting c-Met with a compound recited in claim 12.

20. A method of treatment of a c-Met-mediated disease comprising the administration of a therapeutically effective amount of a compound recited in claim 12 to a patient in need thereof.

21. The method as recited in claim 20 wherein the disease is cancer.

22. A method for achieving an effect in a patient of c-Met inhibition comprising the administration of a therapeutically effective amount of a compound recited in claim 12 to a patient, wherein the effect is selected from the group consisting of Examples 21 through 39.
